SEQUENCE LISTING (17 sequences)

SEQ ID NO: 1 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgcacgtgattttgttggagacgggttaggtacaagctggacgcatgaaaaaaattggaaaaaagcgcataatatcttacttccaagctttagtcagcaggcaatgaaaggctatcatgcgatgatggtcgatatcgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacgacctagtagataaaattattgcagatcgcaaagcaaggggtgaacaaagcgatgatttattaacgcagatgctaaacggaaaagatccagaaacgggtgagccgcttgatgacgggaacattcgctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctcgatattaaagaaactttaacgttaaaacctaaaggcttt

SEQ ID NO: 2 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgcacgtgattttgttggagacgggttaggtacaagctggacgcatgaaaaaaattggaaaaaagcgcataatatcttacttccaagctttagtcagcaggcaatgaaaggctatcatgcgatgatggtcgatatcgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacgacctagtagataaaattattgcagatcgcaaagcaaggggtgaacaaagcgatgatttattaacgcagatgctaaacggaaaagatccagaaacgggtgagccgcttgatgacgggaacattcgctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaatgtttcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctcgatattaaagaaactttaacgttaaaacctaaaggcttt

SEQ ID NO: 3 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcgggtgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgcacgtgattttgttggagacgggttaggtacaagctggacgcatgaaaaaaattggaaaaaagcgcataatatcttacttccaagctttagtcagcaggcaatgaaaggctatcatgcgatgatggtcgataccgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacgacctagtagataaaattattgcagatcgcaaagcaaggggtgaacaaagcgatgatttattaacgcagatgctaaacggaaaagatccagaaacgggtgagccgctcgatgacgggaacattcgctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaatgtttcctgcgttttccctatatgcaaaagaggatacggtgctaggaggagaatatcctttagaaaaaggcgacgaagtaatggttctgattcctcagcttcaccgtgataaaacagtttggggagacgatgtggaggagttccgtccagagcgttttgagaatccaagtgcgattccacagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctcgatattaaagaaactttaacgttaaaacctaaaggcttt

SEQ ID NO: 4 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcgggtgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgcacgtgattttgttggagacgggttaggtacaagctggacgcatgaaaaaaattggaaaaaagcgcataatatcttacttccaagctttagtcagcaggcaatgaaaggctatcatgcgatgatggtcgataccgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacgacctagtagataaaattattgcagatcgcaaagcaaggggtgaacaaagcgatgatttattaacgcagatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaatgtttcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctcgatattaaagaaactttaacgttaaaacctaaaggcttt

SEQ ID NO: 5 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcgggtgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgcacgtgattttgttggagacgggttaggtacaagctggacgcatgaaaaaaattggaaaaaagcgcataatatcttacttccaagctttagtcagcaggcaatgaaaggctatcatgcgatgatggtcgataccgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacgacctagtagataaaattattgcagatcgcaaagcaaggggtgaacaaagcgatgatttattaacgcagatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaatgtctcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgattttgaagatcatacaaactacgagctcgatattaaagaaactttaacgttaaaacctaaaggcttt

SEQ ID NO: 6 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcgggtgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgcacgtgattttgttggagacgggttaggtacaagctggacgcatgaaaaaaattggaaaaaagcgcataatatcttacttccaagctttagtcagcaggcaatgaaaggctatcatgcgatgatggtcgataccgccgtgcagcttgttcaaaagtgggagcgtctaaatgcagatgagcatattgaagtatcggaagacatgacacgtttaacgcttgatacaattggtctttgcggctttaactatcgctttaacagcctttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacgacctagtagataaaattattgcagatcgcaaagcaaggggtgaacaaagcgatgatttattaacgcagatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaatgtctcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgattttgaagatcatacaaactacgagctcgatattaaagaaactttaacgttaaaacctaaaggcttt

SEQ ID NO: 7 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcgggtgaattaggagaaatctttaaattcgaggcgcctggtcgtgtaacgcgcgttttatcaagtcagcgtctaattaaagaagcatgcgatgaatcacgctttgataaaaacttaagtcaagcgcttaaatttgcacgtgattttgttggagacgggttaggtacaagctggacgcatgaaaaaaattggaaaaaagcgcataatatcttacttccaagctttagtcagcaggcaatgaaaggctatcatgcgatgatggtcgataccgccgtgcagcttgttcaaaagtgggagcgtctaaatgcagatgagcatattgaagtatcggaagacatgacacgtttaacgcttgatacaattggtctttgcggctttaactatcgctttaacagcctttaccgagatcagcctcatccatttattataagtatggtccgtgcactggatgaaatgatgaacaagctgcagcgagcaaatccagacgacccagcttatgatgaaaacaagcgccagtttcaagaagatatcaaggtgatgaacgacctagtagataaaattattgcagatcgcaaagcaaggggtgaacaaagcgatgatttattaacgcagatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaatgtctcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgattttgaagatcatacaaactacgagctcgatattaaagaaactttaacgttaaaacctaaaggcttt

SEQ ID NO: 8 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcgggtgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgcacgtgattttgttggagacgggttaggtacaagctggacgcatgaaaaaagttggaaaaaagcgcataatatcttacttccaagctttagtcagcaggcaatgaaaggctatcatgcgatgatggtcgataccgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacggcctagtagataaaattattgcagatcgcaaagcaaggggtgaacaaagcgatgatttattaacgcagatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacacgtattacaaaaagtagcagaagaagcagcacgagttctagtagatcctgttccaagctacaaacaagtcaaacagcttaaatatgtcggcatggtcttaaacgaagcgctgcgcttatggccaatgtctcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgattttgaagatcatacaaactacgagctcgatattaaagaaactttatcgttaaaacctaaaggcttt

SEQ ID NO: 9 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcgggtgaattaggagaaatctttaaattcgaggcgcctggtcgtgtaacgcgctacttatcaagtcagcgtctaattaaagaagcatgcgatgaatcacgctttgataaaaacttaggtcaagcgcttaaatttgcacgtgattttgttggagacgcgttaggtacaagctggacgcatgaaaaaagttggaaaaaagcgcataatatcttacttccaagctttagtcagcaggcaatgaaaggctatcatgcgatgatggtcgataccgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacggcctagtagataaaattattgcagatcgcaaagcaaggggtgaacaaagcgatgatttattaacgcagatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacacgtattacaaaaagtagcagaagaagcagcacgagttctagtagatcctgttccaagctacaaacaagtcaaacagcttaaatatgtcggcatggtcttaaacgaagcgctgcgcttatggccaatgtctcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgattttgaagatcatacaaactacgagctcgatattaaagaaactttatcgttaaaacctaaaggcttt

SEQ ID NO: 10 - DNA, 1332 bp, synthetic construct
  source 1..1332: mol_type = other DNA
acaattaaagaaatgcctcagccaaaaacgtttggagagcttaaaaatttaccgttattaaacacagataaaccggttcaagctttgatgaaaattgcgggtgaattaggagaaatctttaaattcgaggcgcctggtggtgtaacgcgctacttatcaagtcagcgtctaattaaagaagcatgcgatgaatcacgctttgataaaaacttaggtcaagcgcttaaatttgcacgtgattttgttggagacgcgttaggtacaagctggacgcatgaaaaaagttggaaaaaagcgcataatatcttacttccaagctttagtcagcaggcaatgaaaggctatcatgcgatgatggtcgataccgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtttcaagaagatatcaaggtgatgaacggcctagtagataaaattattgcagatcgcaaagcaaggggtgaacaaagcgatgatttattaacgcagatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacacgtattacaaaaagtagcagaagaagcagcacgagttctagtagatcctgttccaagctacaaacaagtcaaacagcttaaatatgtcggcatggtcttaaacgaagcgctgcgcttatggccaatgtctcctgcgttttccctatatgcaaaagaagatacggtgcttggaggagaatatcctttagaaaaaggcgacgaagtaatggttctgattcctcagcttcaccgtgataaaacagtttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgattttgaagatcatacaaactacgagctcgatattaaagaaactttatcgttaaaacctaaaggcttt

SEQ ID NO: 11 - DNA, 50 bp, synthetic construct
  source 1..50: mol_type = other DNA
aactttaagaaggagatatacatatgacaattaaagaaatgcctcagcca

SEQ ID NO: 12 - DNA, 50 bp, synthetic construct
  source 1..50: mol_type = other DNA
tggctgaggcatttctttaattgtcatatgtatatctccttcttaaagtt

SEQ ID NO: 13 - DNA, 40 bp, synthetic construct
  source 1..40: mol_type = other DNA
cttttttagcagactgttcagtgctaggtgaaggaatacc

SEQ ID NO: 14 - DNA, 40 bp, synthetic construct
  source 1..40: mol_type = other DNA
ggtattccttcacctagcactgaacagtctgctaaaaaag

SEQ ID NO: 15 - DNA, 25 bp, synthetic construct
  source 1..25: mol_type = other DNA
gctaaccgcttttttgcacaacatg

SEQ ID NO: 16 - DNA, 24 bp, synthetic construct
  source 1..24: mol_type = other DNA
ttgtgcaaaaaagcggttagctcc

SEQ ID NO: 17 - protein, 6 aa, synthetic construct
HHHHHH

(19) United States
(12) Patent Application Publication
(10) Pub. No.: US 2026/0286403 A1
(43) Pub. Date: Sep. 24, 2026

(54) METHOD OF PREPARING ORGANOSILICON COMPOUNDS WITH SELECTIVE CYTOCHROME P450 VARIANTS AND RELATED COMPOUNDS AND COMPOSITIONS

(71) Applicants: DOW SILICONES CORPORATION, Midland, MI (US); DOW GLOBAL TECHNOLOGIES LLC, Midland, MI (US); CALIFORNIA INSTITUTE OF TECHNOLOGY, Pasadena, CA (US)

(72) Inventors: Ryan MAAR, Midland, MI (US); John ROBERTS, Midland, MI (US); Dimitris KATSOULIS, Midland, MI (US); Jordan REDDEL, Midland, MI (US); Donald ELDRED, Midland, MI (US); Eric JOFFRE, Midland, MI (US); Frances ARNOLD, Pasadena, CA (US); Sabine BRINKMANN-CHEN, Pasadena, CA (US); Nicholas SARAI, Pasadena, CA (US); Tyler FULTON, Pasadena, CA (US); Ryen O'MEARA, Pasadena, CA (US)

(21) Appl. No.: 19/474,517

(22) PCT Filed: Apr. 12, 2024
(86) PCT No.: PCT/US2024/024240

§ 371 Date: Oct. 10, 2025

Related U.S. Application Data
(60) Provisional application No. 63/458,712, filed on Apr. 12, 2023.

(51) Int. Cl. C12P 9/00; C07F 7/18; C07F 7/21

(52) U.S. Cl. CPC C12P 9/00; C07F 7/1872; C07F 7/21

(57) ABSTRACT

A method of preparing an organosilicon compound having at least one silicon-bonded carbinol group and/or at least one silanol group with a cytochrome P450 variant that facilitates the oxidization of a silicon-bonded hydrocarbyl group in the presence of an oxidant. The method includes combining the cytochrome P450 variant, an initial organosilicon compound having at least one silicon-bonded hydrocarbyl group, and a cofactor to give a reaction mixture, and exposing the reaction mixture to an oxidant to oxidize the silicon-bonded hydrocarbyl group of the initial organosilicon compound, thereby preparing the organosilicon compound having at least one silicon-bonded carbinol group and/or at least one silanol group. Cytochrome P450 variants suitable for use in the method are also disclosed, along with methods for engineering and optimizing the same. Nucleic acids encoding the cytochrome P450 variants and compositions, expression vectors, and host cells including the same are also disclosed.

### CROSS-REFERENCE TO RELATED APPLICATIONS

This application claims priority to and all advantages of U.S. Provisional Patent Application No. 63/458,712 filed on 12 Apr. 2023, the content of which is incorporated herein by reference.

### FIELD OF THE INVENTION

The present disclosure relates generally to a catalyst and methods for preparing organosilicon compounds having at least one silicon-bonded carbinol and/or at least one silanol group and, more specifically, to a cytochrome protein variant catalyst that selectively oxidizes a silicon-bonded hydrocarbyl group to at least one silicon-bonded carbinol and/or at least one silanol group in organosilicon compounds, and to methods of using the same.

### DESCRIPTION OF THE RELATED ART

Silicones are polymeric materials used in numerous commercial applications, primarily due to significant advantages they possess over many carbon-based analogues. More particularly referred to as polymerized siloxanes or polysiloxanes, silicones include an inorganic silicon-oxygen backbone chain ( . . . —Si—O—Si—O—Si—O— . . . ) having organic side groups attached to the silicon atoms. Organic side groups may be used to link two or more of these backbones together. By varying the —Si—O— chain lengths, side groups, and cross-linking, silicones can be synthesized with a wide variety of properties and compositions, with silicone networks varying in consistency from liquid to gel to rubber to hard plastic. Silicone and siloxane-based materials are utilized in myriad end use applications and environments, including as components in a wide variety of industrial, home care, and personal care formulations.

Various organosilicon compounds and organopolysiloxanes are utilized as precursors or diluents for preparing many silicone and siloxane-based materials, as well as other products in the silicon industry. Such organosilicon compounds and organopolysiloxanes are also used as synthetic intermediates in organic synthesis, as monomeric building blocks for silicone-organic hybrid materials, and as catalysts for certain reactions.

Such organosilicon compounds and organopolysiloxanes have desirable properties, including stability and longevity under extreme conditions, including exposure to temperature variations, ultraviolet light, etc. However, though these properties are desirable in many end use applications, stability of many organosilicon compounds and organopolysiloxanes results in their accumulation in the environment.

In addition, when such organosilicon compounds and organopolysiloxanes are non-functional, they cannot be easily reacted to form other reaction products, and are more difficult to filter or remove as a result, e.g. from waste or reaction products.

### BRIEF SUMMARY

A method of preparing an organosilicon compound having at least one silicon-bonded carbinol group and/or at least one silanol group is provided. The method comprises combining a cytochrome P450 variant, an initial organosilicon compound, and a cofactor to prepare a reaction mixture. The cytochrome P450 variant facilitates the oxidation of a silicon-bonded hydrocarbyl group to a silicon-bonded carbinol group or a silanol group in the presence of an oxidant, and the initial organosilicon compound has at least one silicon-bonded hydrocarbyl atom. The method also comprises exposing the reaction mixture to an oxidant to oxidize and convert the silicon-bonded hydrocarbyl group of the initial organosilicon compound to a silicon-bonded carbinol group or a silicon-bonded silanol group the organosilicon compound, thereby preparing the organosilicon compound having at least one silicon-bonded carbinol group and/or at least one silanol group.

A method of preparing a reaction product is also provided. The method comprises preparing the reaction mixture, exposing the reaction mixture to an oxidant to oxidize and convert the silicon-bonded hydrocarbyl group to the functional group and give an organosilicon compound having the functional group, and reacting the functional group of the organosilicon compound with a group reactive with the functional group of the organosilicon compound, thereby preparing the reaction product.

Cytochrome P450 variants are also provided. One of the cytochrome P450 variants comprises or is encoded by a nucleic acid sequence of SEQ ID NO:1 or a conservatively modified variant thereof. One of the cytochrome P450 variants comprises or is encoded by a nucleic acid sequence of SEQ ID NO:2 or a conservatively modified variant thereof. Another of the cytochrome P450 variants comprises or is encoded by a nucleic acid sequence of SEQ ID NO:3 or a conservatively modified variant thereof. Another of the cytochrome P450 variants comprises or is encoded by a nucleic acid sequence of SEQ ID NO:4 or a conservatively modified variant thereof. Another of the cytochrome P450 variants comprises or is encoded by a nucleic acid sequence of SEQ ID NO:5 or a conservatively modified variant thereof. Another of the cytochrome P450 variants comprises or is encoded by a nucleic acid sequence of SEQ ID NO:6 or a conservatively modified variant thereof. Another of the cytochrome P450 variants comprises or is encoded by a nucleic acid sequence of SEQ ID NO:7 or a conservatively modified variant thereof. Another of the cytochrome P450 variants comprises or is encoded by a nucleic acid sequence of SEQ ID NO:8 or a conservatively modified variant thereof. Another of the cytochrome P450 variants comprises or is encoded by a nucleic acid sequence of SEQ ID NO:9 or a conservatively modified variant thereof. Another of the cytochrome P450 variants comprises or is encoded by a nucleic acid sequence of SEQ ID NO:10 or a conservatively modified variant thereof.

### DETAILED DESCRIPTION OF THE INVENTION

A method of preparing an organosilicon compound having at least one silicon-bonded carbinol group and/or at least one silanol group is provided. In general, the method comprises exposing a reaction mixture comprising a cytochrome P450 variant and an initial organosilicon compound to an oxidant to prepare the organosilicon compound having at least one silicon-bonded carbinol group and/or at least one silanol group. More specifically, the cytochrome P450 variant is capable of facilitating the oxidation and conversion of at least one silicon-bonded hydrocarbyl group to at least one silicon-bonded carbinol group and/or at least one silanol group, and the initial organosilicon compound comprises at least one silicon-bonded hydrocarbyl group capable of being oxidized to give the organosilicon compound having at least one silicon-bonded carbinol group and/or at least one silanol group.

Notably, although enzymatic manipulations of functional groups proximal to silicon in various organosilicon compounds are known, it is believed that the present invention, as illustrated by the examples and described herein, represents the first biocatalytic transformation of a silicon-bonded hydrocarbyl group (i.e., Si—R, where R is hydrocarbyl) to a silanol group (i.e., Si—OH) or a silicon-bonded carbinol group (i.e., Si—R′—OH, where R′ is also hydrocarbyl). Accordingly, it will be appreciated that certain aspects of the invention described herein in relation to the method may be practiced individually or in various combinations, i.e., without limitation as to any particular end-use, composition, formulation, etc. Such aspects include novel cytochrome P450 variants, materials and compositions relating thereto, as well as various methods of preparing the same.

As will be understood by those of skill in the art, the method and materials described herein relate generally to biocatalysis (i.e., the use of a biological system and/or material to facilitate a chemical reaction), and more specifically to protein-based biocatalysts. For purposes of clarity, certain terms utilized herein are set forth and described below.

The terms “protein,” “peptide,” and “polypeptide” are used interchangeably herein to refer to a polymer of amino acid residues (i.e., a molecule having 2 or more amino acids that are joined together by a peptide bond) or an assembly of multiple polymers of amino acid residues. In some instances, more specific terms may be utilized, such as with reference to one or more particular oligopeptides (i.e., peptides comprising 20 or fewer, optionally 10 or fewer amino acids, e.g. di-, tri-, tetra-, and pentapeptides, etc.) polypeptides (i.e., peptides comprising greater than 10, optionally greater than 20 amino acids), proteins (i.e., organic compounds comprising amino acids linked via peptide bonds in a linear chain and folded into a globular form), enzymes (i.e., functional proteins, optionally comprising cofactors, multiple proteins, etc.), and the like, which may be modified (e.g. naturally and/or synthetically via glycosylation, acetylation, phosphorylation, etc.), branched, etc. Such terms apply to amino acid polymers in which one or more amino acid residues are an artificial chemical mimic of a corresponding naturally occurring amino acid, as well as to naturally occurring (i.e., native) amino acid polymers and non-naturally occurring (i.e., synthetic, engineered, etc.) amino acid polymers. The identity and order of particular amino acid residues in a protein is generally referred to as an “amino acid sequence”.

The term “amino acid” includes both naturally occurring and non-naturally occurring amino acids, as stereoisomers thereof. In this context, a stereoisomer of an amino acid generally refers to a mirror isomer of opposing stereochemistry at the alpha carbon atom, such as an L-stereoisomer (i.e., a left-handed isomer) and a D-stereoisomer (i.e., a right-handed isomer) of the same alpha-amino acid. For example, a stereoisomer of a naturally occurring amino acid (which are L-stereoisomers) refers to the mirror image isomer of the naturally occurring amino acid, i.e., the D-stereoisomer. As will be appreciated from certain examples below, amino acids may be referred to herein by either their commonly known three letter symbols or by the one-letter symbols recommended by the Biochemical Nomenclature Commission of the International Union of Pure and Applied Chemistry and the International Union of Biochemistry (IUPAC-IUB). For example, an L-amino acid may be represented herein by its commonly known three letter symbol (e.g. Arg for L-arginine) or by an upper-case one-letter amino acid symbol (e.g. R for L-arginine). A D-amino acid may be represented herein by its commonly known three letter symbol (e.g. D-Arg for D-arginine) or by a lower-case one-letter amino acid symbol (e.g. r for D-arginine).

Naturally occurring amino acids are those encoded by the genetic code, as well as natural derivative/modifications thereof (e.g. hydroxyproline, γ-carboxyglutamate, O-phosphoserine, etc.). Examples of naturally occurring α-amino acids include, among others, alanine (Ala; A), cysteine (Cys; C), aspartic acid (Asp; D), glutamic acid (Glu; E), phenylalanine (Phe; F), glycine (Gly; G), histidine (His; H), isoleucine (Ile; I), arginine (Arg; R), lysine (Lys; K), leucine (Leu; L), methionine (Met; M), asparagine (Asn; N), proline (Pro; P), glutamine (Gln; Q), serine (Ser; S), threonine (Thr; T), valine (Val; V), tryptophan (Trp; W), and tyrosine (Tyr; Y), and combinations thereof. Likewise, examples of stereoisomers of naturally occurring α-amino acids include D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), and D-tyrosine (D-Tyr). Examples of non-naturally occurring (i.e., unnatural) amino acids include various amino acid analogs and mimetics, as well as synthetic amino acids, in either L- or D-configurations (e.g. N-substituted glycines, and N-methyl amino acids, etc.). Examples of amino acid analogs include unnatural amino acids having the same basic chemical structure as naturally occurring amino acids (i.e., an α-carbon bonded to a hydrogen, a carboxyl group, and an amino group) but a modified side-chain groups, or modified peptide backbones, (e.g. homoserine, norleucine, methionine sulfoxide, methionine methyl sulfonium, etc.). “Amino acid mimetics” refer to chemical compounds that have a structure that is different from the general chemical structure of an amino acid, but that functions in a manner similar to a naturally occurring amino acid.

With respect to the amino acid sequences of proteins, one of skill in the art will recognize that individual substitutions, additions, or deletions that alter, add, and/or delete a single amino acid, or a small percentage of amino acids in the sequence, may be referred to as a “conservative modification” of the amino acid sequence where the alteration results in the substitution of an amino acid with a chemically similar amino acid, particularly where the function of the protein is largely or wholly unchanged. Likewise, a protein having a conservatively modified sequence may be referred to as a “conservatively modified variant” of a wild type or otherwise unmodified protein sequence. Unless otherwise indicated, a particular amino acid sequence is to be understood to implicitly encompass conservatively modified variants in addition to the sequence explicitly indicated.

As will be understood by those of skill in the art, chemically similar amino acids are not limited, and conservative substitution tables setting forth functionally similar amino acids are well known in the art. For example, substitutions may be made wherein one aliphatic amino acid (e.g. G, A, I, L, V, etc.) is substituted with another aliphatic amino acid, where an aliphatic amino acid having a polar-uncharged group (e.g. C, S, T, M, N, Q, etc.) is substituted with another such aliphatic amino acid, where a basic amino acid (e.g. K, R, H, etc.) is substituted for a different basic amino acid, etc. In some instances, a conservative substitution comprises substituting an amino acid with an acidic side chain (e.g. E or D) with an uncharged counterpart (e.g. Q or N, respectively), or vice versa. Each of the following eight groups contains other exemplary amino acids that may be conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine(S) and Threonine (T); and
8) Cysteine (C) and Methionine (M).

The terms “oligonucleotide,” “nucleic acid,” and “polynucleotide” are used interchangeably herein to refer to polymers comprising nucleotides, i.e., deoxyribonucleic acids (DNA) and/or ribonucleic acids (RNA) in either single-, double-, or multi-stranded forms (i.e., single-, double-, and multi-stranded DNA and/or RNA, including genomic DNA, cDNA, DNA-RNA hybrids), as well as polymers comprising purine, pyrimidine, or other nucleotide bases, which may be natural or non-naturally occurring bases (e.g. such as chemically modified, biochemically modified, synthetic, and/or derivatized nucleotide bases). As will be understood by those of skill in the art, a polynucleotide may be described in relation to a peptide encoded thereby, such that the term “nucleotide sequence encoding a peptide” or the like may be used to refer to a segment of DNA involved in producing a peptide chain. Such a segment can include regions preceding and/or following a given coding region (i.e., a leader and/or trailer sequence) involved in the transcription/translation of a gene product or regulation thereof, as well as intervening sequences (introns) between individual coding segments (exons). Accordingly, the term “nucleic acid” and the like may be used interchangeably with gene, cDNA, and mRNA encoded by a gene. Unless specifically limited, the terms also encompass nucleic acids containing known analogs of natural/reference nucleotides that have similar binding properties as the reference nucleic acid, which may be metabolized in a manner similar to naturally occurring nucleotides.

The identity and order of particular nucleotide bases in a polynucleotide is generally referred to as a “nucleic acid sequence”. As with the amino acid sequences described above, unless otherwise indicated, a particular nucleic acid sequence is to be understood to implicitly encompass conservatively modified variants of the sequence in addition to the nucleic acid sequence explicitly indicated. Conservatively modified variants of a polynucleotide generally comprise degenerate codon substitutions, or complementary or orthologous sequences compared to a given wild type or otherwise unmodified nucleic acid sequence. As known in the art, degenerate codon substitutions may be achieved by generating sequences in which the third position of a selected codon is substituted with mixed-base and/or deoxyinosine residues.

The term “homolog,” as used herein with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species.

The terms “homologous” or “homolog” are used herein with reference to similar sequences of polynucleotides and/or nucleic acids. For example, a first protein has “homology” or is “homologous” to a second protein if the amino acid sequence encoded by a gene has a similar amino acid sequence to that of the second gene. Alternatively, a first protein has homology to a second protein if the two proteins have “similar” amino acid sequences. Thus, the term “homologous proteins” is intended to mean that the two proteins have similar amino acid sequences. Similarly, the term “functional homolog” refers to each member of a subgroup of homologs or homologous sequences that share a common functionality, i.e., a primary function for which a protein, gene, sequence, and the like is named and/or utilized. For example, the function of a promoter is to facilitate transcription of a gene or nucleotide sequence and the function of an enzyme is to catalyze a particular chemical reaction or family of chemical reactions. As such, term “functionality” encompasses all reaction rates and all enzymatic efficiencies exhibited by a given protein. In particular embodiments, the homology between two proteins is indicative of its shared ancestry, related by evolution. It will be appreciated that homologs most often have functional, structural, or genomic similarities. For example, in certain embodiments, homologous sequences share at least 70% sequence identity, such as at least 80, alternatively at least 90, alternatively at least 95, alternatively at least 99% sequence identity. Techniques are known by which homologs of an enzyme or gene can readily be cloned using genetic probes and PCR. Identity of cloned sequences as homolog can be confirmed using functional assays and/or by genomic mapping of the genes.

As introduced above, the method utilizes a cytochrome P450 variant capable of oxidizing a silicon-bonded hydrocarbyl group to a silicon-bonded carbinol group or a silanol group. More specifically, the cytochrome P450 variant facilitates the selective oxidation of at least one silicon-bonded hydrocarbyl group (i.e., Si—R, where R is hydrocarbyl) of the initial organosilicon compound to a silanol group (i.e., Si—OH) or a silicon-bonded carbinol group (Si—R′OH, where R′ is a hydrocarbon group), as described in additional detail below, and is otherwise not particularly limited.

As will be understood by those of skill in the art, the term “cytochrome P450” as utilized herein refers to an enzyme classified or otherwise characterized as a member of the cytochrome P450 enzyme family, which is known to comprise a large superfamily of heme-thiolate proteins that typically possess an active site containing an Fe(III)-protoporphyrin IX cofactor (i.e., a heme-iron center) proximally tethered by a highly conserved cysteine thiolate residue. In the resting state, the remaining axial iron coordination site is occupied by a water molecule. However, the heme-iron center is capable of binding molecular oxygen at this axial iron coordination site, giving rise to the native catalytic reactivity.

Cytochrome P450 enzymes are involved in the metabolism of a wide variety of both exogenous and endogenous compounds, and often function as a terminal oxidase in multicomponent electron transfer chains, such as P450-containing monooxygenase systems. Cytochrome P450 enzymes are known to catalyze myriad carbon-centered oxidative transformations, including carbon oxygenations and hydroxylations, epoxidations, oxidative ring couplings, and desaturations. A general chemical mechanism used to rationalize most of the oxidative activity of native cytochrome P450 enzymes involves a perfenyl (FeO³⁺) intermediate and odd-electron chemistry. In particular, the heme-iron center activates molecular oxygen in the presence of an electron source (e.g. nicotinamide adenine dinucleotide (NADH) or nicotinamide adenine dinucleotide phosphate (NADPH), such as from an adjacent fused reductase domain, an accessory cytochrome P450 reductase enzyme, etc.) to generate a molecule of water and an iron(IV)-oxo porphyrin radical cation intermediate conventionally known as “P450 Compound 1”. More specifically, after induction of a first electron transfer (e.g. via substrate binding), molecular oxygen binds to the ferrous heme center to give a dioxygen adduct (e.g. Fe—O₂). The Fe—O₂ adduct is reduced via a second electron transfer to give a peroxo intermediate, which undergoes rapid dipronation (i.e., two protonations) to release water and give the iron(IV) oxo intermediate (i.e., P450 Compound 1). Alternatively, P450 Compound 1 can be formed with a hydrogen peroxide shunt which can bypass the need for an electronic source. The highly reactive iron(IV) oxo intermediate then reacts with a substrate at a C—H or C═C bond (e.g. via abstraction of a hydrogen atom or electron, followed by oxygen rebound, rearrangement, etc.) to affect an oxidative transformation (e.g. a hydroxylation, epoxidation etc.).

As understood in the art, both genes encoding cytochrome P450 enzymes, as well as the enzymes themselves, may be designated according to a common naming convention utilizing the root symbol “CYP” indicating the superfamily, followed by: 1) a number indicating the gene family; 2) a capital letter indicating the subfamily; and 3) a numeral indicating an individual gene. For example, the gene designated “CYP102A1” encodes the enzyme CYP102A1 (also known as cytochrome P450 BM3), which is isolated from soil bacterium Bacillus megaterium and facilitates the NADPH-dependent hydroxylation of long-chain fatty acids at the ω-1 through ω-3 positions. Typically, members of a CYP family share at least 40% amino acid identity, while members of subfamilies share at least 55% amino acid identity.

As introduced above, the method utilizes a cytochrome P450 variant. The term “variant”, as used herein in the context of a “protein variant” or “enzyme variant” (e.g. the cytochrome P450 variant) describes a protein or enzyme comprising at least one amino acid mutation (e.g. a substitution) with respect to a wild-type version of the protein/enzyme, including chimeric enzymes comprising recombined sequences or blocks of amino acids from two, three, or more different proteins. However, it is to be understood that certain protein variants need not be prepared via purposeful mutagenesis, but may instead be a natural enzyme that exhibits a desired activity and/or substrate specificity (i.e., selective silane oxidation) that is not natively exhibited by one or more homologous wild-type enzymes. As such, it is to be understood that the term “cytochrome P450 variant” as used herein encompasses the particular cytochrome P450 variants designated by given sequences and provided according to some aspects of this disclosure, as well as certain wild-type cytochrome P450 variants suitable for use in the method, which are described in further detail below. It is also to be appreciated that the cytochrome P450 variant may comprise, or be, a fragment of a cytochrome P450 enzyme that exhibits the activity and/or substrate specificity required to prepare the organosilicon compound having at least one silicon-bonded carbinol group and/or at least one silanol group.

The cytochrome P450 variant utilized in the method is capable of oxidizing a silicon-bonded hydrocarbyl group of an initial organosilicon compound to give a silicon-bonded carbinol group and/or a silanol group in an organosilicon compound, and is otherwise not particularly limited. As such, examples of cytochrome P450 variants suitable for use in the method include those which facilitate the selective oxidation of a silicon-bonded hydrocarbyl group (i.e., Si—R, where R is hydrocarbyl) of the initial organosilicon compound to a silicon-bonded carbinol group (i.e., Si—R′—OH, where R′ is a hydrocarbon group derived from oxidation of R) and/or a silanol group (i.e., Si—OH), as described in additional detail below.

The cytochrome P450 variant is typically an engineered variant of P450 BM3 (CYP102A1) protein. For example, in some such embodiments, the cytochrome P450 variant is a mutant of cytochrome P450 BM3 comprising a structural mutation, such as an amino acid substitution, deletion, duplication, and/or insertion. In particular embodiments, the structural mutation is an amino acid substitution.

As understood by those of skill in the art, cytochrome P450 BM3 is a self-sufficient 118-kDa monooxygenase having a flavin adenine dinucleotide (FAD)- and flavin mononucleotide (FMN)-containing NADPH-dependent reductase domain fused to the C-terminus of a heme domain. Nucleotide and amino acid sequences for cytochrome P450 BM3 may be obtained from public databases, such as the GenBank database maintained by the U.S. National Center for Biotechnology Information (NCBI) under the International Nucleotide Sequence Database Collaboration (INSDC), or the UniProt database maintained by the UniProt Consortium under accession number P14779.

In some embodiments, the cytochrome P450 variant comprises a nucleic acid sequence having at least 70% identity to the nucleic acid sequence set forth in SEQ ID NO:1, such as at least 75, alternatively at least 80, alternatively at least 85, alternatively at least 90, alternatively at least 95% identity to the nucleic acid sequence set forth in SEQ ID NO:1. In particular embodiments, the cytochrome P450 variant comprises a nucleic acid sequence having greater than 95%, alternatively greater than 96%, alternatively greater than 97%, alternatively greater than 98%, alternatively greater than 99%, alternatively 100% identical to the nucleic acid sequence set forth in SEQ ID NO:1. When the cytochrome P450 variant has the nucleic acid sequence set forth in SEQ ID NO:1, the cytochrome P450 variant comprises a V79 mutation relative to the nucleic acid sequence of cytochrome P450 BM3, typically a V79A mutation. In these or other embodiments, the cytochrome P450 variant of SEQ IQ NO:1 comprises an A83 mutation relative to the nucleic acid sequence of cytochrome P450 BM3, typically an A83V mutation. In these or other embodiments, the cytochrome P450 variant of SEQ IQ NO:1 comprises an F88 mutation relative to the nucleic acid sequence of cytochrome P450 BM3, typically an F88G mutation. In these or other embodiments, the cytochrome P450 variant of SEQ IQ NO:1 comprises an P143 mutation relative to the nucleic acid sequence of cytochrome P450 BM3, typically a P143S mutation. In these or other embodiments, the cytochrome P450 variant of SEQ IQ NO:1 comprises a T176 mutation relative to the nucleic acid sequence of cytochrome P450 BM3, typically a T1761 mutation. In these or other embodiments, the cytochrome P450 variant of SEQ IQ NO:1 comprises an A185 mutation relative to the nucleic acid sequence of cytochrome P450 BM3, typically an A185V mutation. In these or other embodiments, the cytochrome P450 variant of SEQ IQ NO:1 comprises an S227 mutation relative to the nucleic acid sequence of cytochrome P450 BM3, typically an S227R mutation. In these or other embodiments, the cytochrome P450 variant of SEQ IQ NO:1 comprises an H237 mutation relative to the nucleic acid sequence of cytochrome P450 BM3, typically an H237Q mutation. In these or other embodiments, the cytochrome P450 variant of SEQ IQ NO:1 comprises an E253 mutation relative to the nucleic acid sequence of cytochrome P450 BM3, typically an E253G mutation. In these or other embodiments, the cytochrome P450 variant of SEQ IQ NO:1 comprises an A291 mutation relative to the nucleic acid sequence of cytochrome P450 BM3, typically an A291V mutation. In these or other embodiments, the cytochrome P450 variant of SEQ IQ NO:1 comprises an L354 mutation relative to the nucleic acid sequence of cytochrome P450 BM3, typically an L354V mutation. In these or other embodiments, the cytochrome P450 variant of SEQ IQ NO:1 comprises an 1367 mutation relative to the nucleic acid sequence of cytochrome P450 BM3, typically an 1367V mutation. In these or other embodiments, the cytochrome P450 variant of SEQ IQ NO:1 comprises an E443 mutation relative to the nucleic acid sequence of cytochrome P450 BM3, typically an E443K mutation. In these or other embodiments, the cytochrome P450 variant of SEQ IQ NO:1 comprises an F108 silent mutation relative to the nucleic acid sequence of cytochrome P450 BM3, typically an F108F silent mutation. In these or other embodiments, the cytochrome P450 variant of SEQ IQ NO:1 comprises an E273 silent mutation relative to the nucleic acid sequence of cytochrome P450 BM3, typically an E273E silent mutation. In a specific embodiment, the cytochrome P450 variant of SEQ IQ NO:1 includes V79A, A83V, F88G, P143S, T1761, A185V, S227R, H237Q, E253G, A291V, L354V, 1367V, E443K mutations, and F108F and E273E silent mutations, relative to the nucleic acid sequence of cytochrome P450BM3.

In specific embodiments, the cytochrome P450 variant comprises a T328 mutation relative to the nucleic acid sequence of SEQ ID NO:1. For example, in some such embodiments, the T328 mutation is a T328M mutation. In these or other embodiments, the cytochrome P450 variant comprises an A329 mutation relative to the nucleic acid sequence of SEQ ID NO:1. For example, in some such embodiments, the A329 mutation is an A329F mutation. In these or other embodiments, the cytochrome P450 variant comprises an 1454 silent mutation relative to the nucleic acid sequence of SEQ ID NO:1. For example, in some such embodiments, the 1454 silent mutation is an 1454| silent mutation. In particular embodiments, the cytochrome P450 variant comprises T328M and A329F mutations relative to the nucleic acid sequence of SEQ ID NO:1 along with 1454 silent mutations relative to the nucleic acid sequence of SEQ ID NO:1. In some such embodiments, the cytochrome P450 variant comprises a nucleic acid sequence that having at least 70% identity to the nucleic acid sequence set forth in SEQ ID NO:2, such as at least 75, alternatively at least 80, alternatively at least 85, alternatively at least 90, alternatively at least 95% identity to the nucleic acid sequence set forth in SEQ ID NO:2. In particular such embodiments, the cytochrome P450 variant comprises a nucleic acid sequence having greater than 98%, alternatively greater than 99%, alternatively 100% identical to the nucleic acid sequence set forth in SEQ ID NO:2.

In specific embodiments, the cytochrome P450 variant comprises a D35 mutation relative to the nucleic acid sequence of SEQ ID NO:2. For example, in some such embodiments, the D35 mutation is a D35G mutation. In these or other embodiments, the cytochrome P450 variant comprises an 1123 mutation relative to the nucleic acid sequence of SEQ ID NO:2. For example, in some such embodiments, the 1123 mutation is an 1123T mutation. In these or other embodiments, the cytochrome P450 variant comprises an L250 silent mutation relative to the nucleic acid sequence of SEQ ID NO:2. For example, in some such embodiments, the L250 silent mutation is an L250L silent mutation. In these or other embodiments, the cytochrome P450 variant comprises an E338 silent mutation relative to the nucleic acid sequence of SEQ ID NO:2. For example, in some such embodiments, the E338 silent mutation is an E338E silent mutation. In these or other embodiments, the cytochrome P450 variant comprises an L342 silent mutation relative to the nucleic acid sequence of SEQ ID NO:2. For example, in some such embodiments, the L342 silent mutation is an L342L silent mutation. In these or other embodiments, the cytochrome P450 variant comprises an E381 silent mutation relative to the nucleic acid sequence of SEQ ID NO:2. For example, in some such embodiments, the E381 silent mutation is an E381E silent mutation. In these or other embodiments, the cytochrome P450 variant comprises a P387 silent mutation relative to the nucleic acid sequence of SEQ ID NO:2. For example, in some such embodiments, the P387 silent mutation is a P387P silent mutation. In particular embodiments, the cytochrome P450 variant comprises both D35G and 1123T mutations, and L250L, E388E, L342L, E381E, and P387P silent mutations relative to the nucleic acid sequence of SEQ ID NO:2. In some such embodiments, the cytochrome P450 variant comprises a nucleic acid sequence that having at least 70% identity to the nucleic acid sequence set forth in SEQ ID NO:3, such as at least 75, alternatively at least 80, alternatively at least 85, alternatively at least 90, alternatively at least 95% identity to the nucleic acid sequence set forth in SEQ ID NO:3. In particular such embodiments, the cytochrome P450 variant comprises a nucleic acid sequence having greater than 95%, alternatively greater than 96%, alternatively greater than 97%, alternatively greater than 98%, alternatively greater than 99%, alternatively 100% identical to the nucleic acid sequence set forth in SEQ ID NO:3.

In specific embodiments, the cytochrome P450 variant comprises a G353 mutation relative to the nucleic acid sequence of SEQ ID NO:3. For example, in some such embodiments, the D35 mutation is a D353E mutation. In these or other embodiments, the cytochrome P450 variant comprises an L250 silent mutation relative to the nucleic acid sequence of SEQ ID NO:3. For example, in some such embodiments, the L250 silent mutation is an L250L silent mutation. In these or other embodiments, the cytochrome P450 variant comprises an E338 silent mutation relative to the nucleic acid sequence of SEQ ID NO:3. For example, in some such embodiments, the E338 silent mutation is an E338E silent mutation. In these or other embodiments, the cytochrome P450 variant comprises an L342 silent mutation relative to the nucleic acid sequence of SEQ ID NO:3. For example, in some such embodiments, the L342 silent mutation is an L342L silent mutation. In these or other embodiments, the cytochrome P450 variant comprises an E381 silent mutation relative to the nucleic acid sequence of SEQ ID NO:3. For example, in some such embodiments, the E381 silent mutation is an E381E silent mutation. In these or other embodiments, the cytochrome P450 variant comprises a P387 silent mutation relative to the nucleic acid sequence of SEQ ID NO:3. For example, in some such embodiments, the P387 silent mutation is a P387P silent mutation. In particular embodiments, the cytochrome P450 variant comprises a D353 E mutation, and L250L, E388E, L342L, E381E, and P387P silent mutations relative to the nucleic acid sequence of SEQ ID NO:3. In some such embodiments, the cytochrome P450 variant comprises a nucleic acid sequence that having at least 70% identity to the nucleic acid sequence set forth in SEQ ID NO:4, such as at least 75, alternatively at least 80, alternatively at least 85, alternatively at least 90, alternatively at least 95% identity to the nucleic acid sequence set forth in SEQ ID NO:4. In particular such embodiments, the cytochrome P450 variant comprises a nucleic acid sequence having greater than 95%, alternatively greater than 96%, alternatively greater than 97%, alternatively greater than 98%, alternatively greater than 99%, alternatively 100% identical to the nucleic acid sequence set forth in SEQ ID NO:4

In specific embodiments, the cytochrome P450 variant comprises an F329 mutation relative to the nucleic acid sequence of SEQ ID NO:4. For example, in some such embodiments, the F329 mutation is an F329S mutation. In these or other embodiments, the cytochrome P450 variant comprises a D423 silent mutation relative to the nucleic acid sequence of SEQ ID NO:4. For example, in some such embodiments, the D423 silent mutation is a D423D silent mutation. In particular embodiments, the cytochrome P450 variant comprises an F329 mutation a D432D silent mutation relative to the nucleic acid sequence of SEQ ID NO:4. In some such embodiments, the cytochrome P450 variant comprises a nucleic acid sequence that having at least 70% identity to the nucleic acid sequence set forth in SEQ ID NO:5, such as at least 75, alternatively at least 80, alternatively at least 85, alternatively at least 90, alternatively at least 95% identity to the nucleic acid sequence set forth in SEQ ID NO:5. In particular such embodiments, the cytochrome P450 variant comprises a nucleic acid sequence having greater than 95%, alternatively greater than 96%, alternatively greater than 97%, alternatively greater than 98%, alternatively greater than 99%, alternatively 100% identical to the nucleic acid sequence set forth in SEQ ID NO:5

In specific embodiments, the cytochrome P450 variant comprises an F166 mutation relative to the nucleic acid sequence of SEQ ID NO:5. For example, in some such embodiments, the F166 mutation is an F166L mutation. In some such embodiments, the cytochrome P450 variant comprises a nucleic acid sequence that having at least 70% identity to the nucleic acid sequence set forth in SEQ ID NO:6, such as at least 75, alternatively at least 80, alternatively at least 85, alternatively at least 90, alternatively at least 95% identity to the nucleic acid sequence set forth in SEQ ID NO:6. In particular such embodiments, the cytochrome P450 variant comprises a nucleic acid sequence having greater than 95%, alternatively greater than 96%, alternatively greater than 97%, alternatively greater than 98%, alternatively greater than 99%, alternatively 100% identical to the nucleic acid sequence set forth in SEQ ID NO:6.

In specific embodiments, the cytochrome P450 variant comprises a Y52 mutation relative to the nucleic acid sequence of SEQ ID NO:6. For example, in some such embodiments, the Y52 mutation is a Y52V mutation. In these or other embodiments, the cytochrome P450 variant comprises a V185 mutation relative to the nucleic acid sequence of SEQ ID NO:6. For example, in some such embodiments, the V185 mutation is a V185M mutation. In particular embodiments, the cytochrome P450 variant comprises both Y52V and V185M mutations relative to the nucleic acid sequence of SEQ ID NO:6. In some such embodiments, the cytochrome P450 variant comprises a nucleic acid sequence that having at least 70% identity to the nucleic acid sequence set forth in SEQ ID NO:7, such as at least 75, alternatively at least 80, alternatively at least 85, alternatively at least 90, alternatively at least 95% identity to the nucleic acid sequence set forth in SEQ ID NO:7. In particular such embodiments, the cytochrome P450 variant comprises a nucleic acid sequence having greater than 95%, alternatively greater than 96%, alternatively greater than 97%, alternatively greater than 98%, alternatively greater than 99%, alternatively 100% identical to the nucleic acid sequence set forth in SEQ ID NO:7.

In specific embodiments, the cytochrome P450 variant comprises an N96 mutation relative to the nucleic acid sequence of SEQ ID NO:7. For example, in some such embodiments, the N96 mutation is an N95S mutation. In these or other embodiments, the cytochrome P450 variant comprises a D215 mutation relative to the nucleic acid sequence of SEQ ID NO:7. For example, in some such embodiments, the D215 mutation is a D215G mutation. In these or other embodiments, the cytochrome P450 variant comprises a T439 mutation relative to the nucleic acid sequence of SEQ ID NO:7. For example, in some such embodiments, the T439 mutation is an T439S mutation. In these or other embodiments, the cytochrome P450 variant comprises an H286 silent mutation relative to the nucleic acid sequence of SEQ ID NO:7. In particular embodiments, the cytochrome P450 variant comprises N96S, D215G, and T439S mutations, and an H286H silent mutation, relative to the nucleic acid sequence of SEQ ID NO:7. In some such embodiments, the cytochrome P450 variant comprises a nucleic acid sequence that having at least 70% identity to the nucleic acid sequence set forth in SEQ ID NO:8, such as at least 75, alternatively at least 80, alternatively at least 85, alternatively at least 90, alternatively at least 95% identity to the nucleic acid sequence set forth in SEQ ID NO:8. In particular such embodiments, the cytochrome P450 variant comprises a nucleic acid sequence having greater than 98%, alternatively greater than 99%, alternatively 100% identical to the nucleic acid sequence set forth in SEQ ID NO:8.

In specific embodiments, the cytochrome P450 variant comprises an S73 mutation relative to the nucleic acid sequence of SEQ ID NO:8. For example, in some such embodiments, the S73 mutation is an S73G mutation. In these or other embodiments, the cytochrome P450 variant comprises a G86 mutation relative to the nucleic acid sequence of SEQ ID NO:8. For example, in some such embodiments, the G86 mutation is a V1G86A mutation. In particular embodiments, the cytochrome P450 variant comprises both S73G and G86A mutations relative to the nucleic acid sequence of SEQ ID NO:8. In some such embodiments, the cytochrome P450 variant comprises a nucleic acid sequence that having at least 70% identity to the nucleic acid sequence set forth in SEQ ID NO:9, such as at least 75, alternatively at least 80, alternatively at least 85, alternatively at least 90, alternatively at least 95% identity to the nucleic acid sequence set forth in SEQ ID NO:9. In particular such embodiments, the cytochrome P450 variant comprises a nucleic acid sequence having greater than 95%, alternatively greater than 96%, alternatively greater than 97%, alternatively greater than 98%, alternatively greater than 99%, alternatively 100% identical to the nucleic acid sequence set forth in SEQ ID NO:9.

In specific embodiments, the cytochrome P450 variant comprises an R48 mutation relative to the nucleic acid sequence of SEQ ID NO:9. For example, in some such embodiments, the R48 mutation is an R48G mutation. In some such embodiments, the cytochrome P450 variant comprises a nucleic acid sequence that having at least 70% identity to the nucleic acid sequence set forth in SEQ ID NO:10, such as at least 75, alternatively at least 80, alternatively at least 85, alternatively at least 90, alternatively at least 95% identity to the nucleic acid sequence set forth in SEQ ID NO:10. In particular such embodiments, the cytochrome P450 variant comprises a nucleic acid sequence having greater than 95%, alternatively greater than 96%, alternatively greater than 97%, alternatively greater than 98%, alternatively greater than 99%, alternatively 100% identical to the nucleic acid sequence set forth in SEQ ID NO:10.

It will be understood by those of skill in the art that, with respect to a particular nucleic acid sequence of a cytochrome P450, the conventional numbering utilized to identify/signify a particular amino acid residue disregards the initial methionine residue (e.g. encoded by the start codon), such that the first residue following the initial methionine is the first numbered residue in the sequence. As such, with respect to the mutations above, it will likewise be appreciated that such residues may be assigned or otherwise designated based on the conventional numbering of the cytochrome P450 variant rather than a sequential numbering, such that, by way of example, V79, A83, F88, P143, and T176 mutations may be instead referred to as V78, A82, F87, P142 and T175 mutations, respectively. Further still, one of skill in the art readily appreciates that SEQ ID NO:1 to SEQ ID NO:10 are nucleic acid sequences that encode an amino acid sequence, and that the nucleic acid sequences of SEQ ID NO:1 to SEQ ID NO:10 can be translated to unique amino acid sequences.

It is also to be appreciated that the cytochrome P450 variant may comprise mutations (e.g. substitutions, etc.) at one or more residues other than those described above, as alternative or additional mutations. In general, residues suitable for mutations to prepare cytochrome P450 variants suitable for the method will be determined by those of skill in the art (e.g. based on the particular wild-type enzyme being modified, potential initial organosilicon compounds to be oxidized, conditions to be utilized, etc.), and generally include conserved residues capable of influencing the reaction characteristics of the enzyme (e.g. reactivity of the heme-iron center, selectivity, solvent tolerance, and/or cofactor dependence of the enzyme, etc.). For example, in some embodiments, the cytochrome P450 variant comprises a mutation allowing for the incorporation of non-native cofactors, such as alternative heme cofactors (e.g. protoporphyrin IX or other porphyrin molecules containing metals other than iron, such as cobalt, rhodium, copper, ruthenium, iridium, and manganese, etc.) and/or alternative reducing cofactors (e.g. NADH vs. NADPH, etc.). Such residues can be identified using any technique known in the art, including crystallographic studies, phylogenetic studies, mutagenesis studies, etc., for which procedures are well known.

Mutations may be introduced into the sequence of the cytochrome P450 variant using standard gene synthesis and/or cloning techniques such as directed mutagenesis techniques, random mutagenesis techniques, etc., as well as various combinations thereof. Examples of such techniques include error-prone polymerase chain reaction (PCR), cassette mutagenesis, oligonucleotide-directed mutagenesis, parallel PCR, random mutagenesis with random fragmentation and reassembly via mutual priming, chemical mutagenesis, irradiation, DNA shuffling, and the like, as well as modifications and/or combinations thereof. In certain embodiments, the cytochrome P450 variant is prepared using site-directed mutagenesis (i.e., introducing specific nucleotide changes at pre-determined locations), such as via PCR site-directed mutagenesis, cassette mutagenesis, whole plasmid mutagenesis, Kunkel's method, or the like, or a combination thereof. Certain techniques will be selected based on the particular cytochrome P450 variant being prepared. For example, in certain embodiments, directed mutagenesis techniques may be used to selectively substitute one or more of the conserved residues described above. In these or other embodiments, one or more of the mutagenesis techniques above may also be employed under low-fidelity polymerization conditions introduce random point mutations over a long sequence, mutagenize a mixture of fragments of unknown sequences, etc. As such, it will be appreciated that the above techniques are not limiting, and other techniques may also be utilized.

In certain embodiments, the cytochrome P450 variant is engineered using directed evolution. In such embodiments, directed evolution is utilized to optimize the cytochrome P450 variant, i.e., by generating a saturation mutagenesis library (e.g. via single-site-saturation mutagenesis, double-site-saturation mutagenesis, etc.) and selecting cytochrome P450 variants exhibiting improved activity upon screening, as described in additional detail below.

As will be understood by those of skill in the art, saturation mutagenesis is a technique utilized to introduce random mutations at predetermined locations in an encoded protein. More specifically, saturation mutagenesis typically utilizes artificial gene sequences synthesized using one or more primers containing degenerate codons for introducing variability into the position(s) being optimized. Each of three positions within a degenerate codon encodes a base such as adenine (A), cytosine (C), thymine (T), or guanine (G), or a degenerate position such as K (representing G and T), M (representing A and C), R (representing A and G), S (representing C and G), W (representing A and T), Y (representing C and T), B (representing C, G, and T), D (representing A, G, and T), H (representing A, C, and T), V (representing A, C, and G), or N (representing A, C, G, and T). For example, the degenerate codon “NDT” includes 12 codons (i.e., N=[A, C, G, T]; D=[A, G, T]; T=[T]), which collectively encode 12 amino acids (Phe, Leu, Ile, Val, Tyr, His, Asn, Asp, Cys, Arg, Ser, and Gly). Likewise, the degenerate codon NNN is considered “fully randomized,” as it includes all 64 codons and encodes all 20 naturally occurring amino acids. It will be appreciated that certain amino acids are encoded by more codons than others, such that the exact ratio of encoded amino acids in a given degenerate codon will not be equal. Additionally, degenerate codons are typically selected to minimize the presence of stop codons. For example, restricted degenerate codons “NNK” and “NNS” may be utilized to encode the same number of amino acids as “NNN” (i.e., all 20 natural amino acids), but with a greatly reduced content of encoded stop codons. As such, in certain embodiments, a mixture of degenerate primers may be utilized to achieve desired parameters including redundancy and stop codon content, as well as the representation of select chemical and/or physical characteristics of the amino acids encoded, such as charge, size, electronics, polarity, hydrophilicity, and hydrophobicity. Such mixtures may comprise any number of different degenerate primers in any ratio. Considerations and methods for choosing optimal combinations of degenerate primers will be known to one of skill in the art. For example, computational tools for selecting particular degenerate codons and are known and may be utilized to control the corresponding encoded amino acids. Specific primers suitable for introducing particular mutations described above are provided herein in the examples below.

It is to be understood that the parent proteins/enzymes to be evolved can be a wild-type protein or enzyme, or a variant, mutant, etc. In general, parent proteins are selected from cytochrome P450 proteins such as cytochrome P450 BM3. As such, parent polynucleotides for use in creating the mutagenesis library, as well as entire vectors containing nucleic acids encoding the parent protein of interest, may be commercially available, and thus can be prepared, purchased, or otherwise obtained from any suitable commercial or non-commercial source.

Once prepared, (e.g. via introducing one or more mutations into a target gene encoding the parent cytochrome P450, such as with one or more of the techniques described above), evolved polynucleotides are cloned into a suitable vector and introduced into a suitable host cell (e.g. via transformation, transfection, infection, etc.) for expression, according to methods well known in the art. Appropriate vectors, host cells, and techniques will be readily selected by one of skill in the art. Examples of suitable vectors generally include various plasmids and viruses known to be compatible with host cells that express oxidation enzymes or oxygenases. Examples of suitable host cells generally include bacterial cells (e.g. from Escherichia coli (E. coli), Bacillus, Pseudomonas, etc.), yeast cells (e.g. from Saccharomyces cerevisiae, etc.), fungal cells (e.g. from Aspergillus), insect cells, etc. In certain embodiments, plant and/or other animal cells (e.g. mammalian, human, etc.) may also be utilized. Such host cells may be transformed, transfected or infected as appropriate by any suitable method, including electroporation, chemical-mediated DNA uptake, fungal infection, viral infection, microinjection, microprojectile transformation, and the like, or other techniques known in the art.

Once expressed, evolved cytochrome P450 variants are then tested/screened (e.g. in vivo or in vitro via combination with the organosilicon compound, as described below, with silane oxidation/silanol formation monitored via chromatographic and/or spectroscopic methods) to identify particular variants exhibiting a desired activity or property and, optimally, activity greater (i.e., more beneficial) than the parent cytochrome P450 protein. Any such identified variants may then isolated, purified, and/or characterized as desired, and optionally subjected to assays designed to further test functional activity, etc. It will be appreciated that identified variants may also be utilized in further rounds of directed evolution, i.e., as a parent protein from which a subsequent generation of cytochrome P450 variants is prepared (e.g. via the procedures described above). Various aspects of the directed evolution techniques suitable for use in engineering and/or optimizing the cytochrome P450 variants will be better understood in view of certain procedures set form in the Examples below.

As will be appreciated from the description of the directed evolution process above, as well as the additional description below, a nucleic acid (i.e., a nucleic acid molecule) encoding the cytochrome P450 variant is also provided herein. The nucleic acid molecule may be a DNA molecule or an RNA molecule, and in any form (e.g. such as any of the forms described above) suitable for use in preparing the cytochrome P450 variant. As such, the nucleic acid molecule may encode the cytochrome P450 variant or a precursor thereof, e.g. a pro- or pre-proform of the cytochrome P450 variant, optionally comprising a signal sequence or other heterologous amino acid portion(s) (e.g. a tag) for secretion and/or purification. For example, an affinity tag (e.g. a His6-tag (SEQ ID NO: 17), a glutathione S-transferase (GST), etc.) may be added to the N- and/or C-terminus of a cytochrome P450 variant protein expressed from an expression vector utilizing the nucleic acid in order to facilitate protein purification.

In certain embodiments, the nucleic acid molecule is generated via gene synthesis (i.e., is a synthetic nucleic acid). In some such embodiments, the synthetic nucleic acid is codon-optimized for expression. For example, in certain embodiments, the synthetic nucleic acid may be engineered to lack certain internal restriction endonuclease sites. Likewise, in certain embodiments, the nucleic acid molecule may comprise, or otherwise may be operatively linked to, an expression control sequence, i.e., a sequence allowing expression of the nucleic acid molecule in a desired host cell. Examples of suitable expression control sequences and vectors are known in the art.

Accordingly, a non-human organism transformed or transfected with the nucleic acid molecule (i.e., a transgenic organism) is also provided, and may be prepared by known methods of homologous recombination or other techniques for genetic transfer, such as any of those described herein.

For example, the nucleic acid molecule may be located on a vector. As such, an expression vector comprising a nucleic acid sequence that encodes the cytochrome P450 variant is also provided. In general, the expression vector is not limited, and may be a viral vector, a plasmid, a phage, a phagemid, a cosmid, a fosmid, a bacteriophage (e.g. a bacteriophage P1-derived vector (PAC)), a baculovirus vector, a yeast plasmid, an artificial chromosome (e.g. a bacterial artificial chromosome (BAC), a yeast artificial chromosome (YAC), a mammalian artificial chromosome (MAC), a human artificial chromosome (HAC), etc.), or other such vectors capable of facilitating the expression of the cytochrome P450 variant, which will be readily apparent to one of skill in the art.

The expression vector may include chromosomal, non-chromosomal, and/or synthetic DNA sequences. In certain embodiments, the expression vector comprises a promotor operably linked to nucleic acid sequence that encodes the cytochrome P450 variant. In such embodiments, the promoter is not particularly limited, and may be selected from viral, bacterial, archaeal, fungal, insect, plant, and/or mammalian promoters. In certain embodiments, the promoter is a constitutive promoter. In some embodiments, the promoter is an inducible promoter. In other embodiments, the promoter is a tissue-specific, environmentally regulated, and/or developmentally regulated.

Examples of expression vectors include pCWori vectors, pET vectors (e.g. pET22), pQE vectors, pBluescript vectors, pNH vectors, lambda-ZAP vectors, pKLAC1 vectors, pKLAC2 vectors, pMT vectors, BacPak baculoviral vectors, pSyn_1 vectors, pCR-TOPO vectors, pChlamy_1 vectors, pAdeno-X adenoviral vectors, and pBABE retroviral vectors, and the like, which are available from various commercial suppliers. Additional examples of expression vectors include ptrc99a, pKK223-3, pDR540, pRIT2T, pRSET, pGEM1, pMAL, pBR322 (i.e., ATCC37017), pXT1, pSG5, pSVK3, pBPV, pMSG, pSVLSV40, pcDNA3.3, pcDNA4/TO, pcDNA6/TR, pLenti6/TR, and the like, as well as derivatives and modifications thereof. It will be appreciated that any other vector replicable and viable in the host cell may also be utilized.

The cytochrome P450 variant may be expressed in whole cells, such as bacterial cells, archaeal cells, yeast cells, fungal cells, insect cells, plant cells, mammalian cells, etc. Examples of bacterial host cells include BL21 E. coli, DE3 strain E. coli, E. coli M15, DH5a, DH10β, HB101, T7 Express Competent E. coli (NEB), B. subtilis cells, Pseudomonas fluorescens cells, and cyanobacterial cells such as Chlamydomonas reinhardtii cells and Synechococcus elongates cells. Examples of archaeal host cells include Pyrococcus furiosus, Metallosphaera sedula, Thermococcus litoralis, Methanobacterium thermoautotrophicum, Methanococcus jannaschii, Pyrococcus abyssi, Sulfolobus solfataricus, Pyrococcus woesei, and Sulfolobus shibatae. Examples of fungal host cells include yeast cells from the genera Saccharomyces (e.g. S. cerevisiae), Pichia (e.g. P. Pastoris), Kluyveromyces (e.g. K. lactis), Hansenula, and Yarrowia, as well as filamentous fungal cells from the genera Aspergillus, Trichoderma, and Myceliophthora. Examples of insect host cells include Sf9 cells from Spodoptera frugiperda, Sf21 cells from Spodoptera frugiperda, Hi-Five cells, BTI-TN-5B1-4 Trichophusia ni cells, as well as Schneider 2 (S2) and Schneider 3 (S3) cells from Drosophila melanogaster. Examples of mammalian host cells include HEK293 cells, Hela cells, CHO cells, COS cells, Jurkat cells, NSO hybridoma cells, baby hamster kidney (BHK) cells, MDCK cells, and NIH-3T3 fibroblast cells. Examples of plant host cells include those from tobacco, tomato, potato, maize, rice, lettuce, and spinach plants, as well as other plant cells having short generation times and/or yield reasonable biomass with standard cultivation techniques. It will be appreciated that these host cells also exemplify the non-human organism comprising the nucleic acid molecule according to certain embodiments, as described above.

In some embodiments, the cytochrome P450 variant exhibits enhanced activity compared to a corresponding wild-type cytochrome P450 protein, with respect to the silicon oxidization of the method. For example, in certain embodiments, the cytochrome P450 variant exhibits an activity of at least 1.5 times higher than the corresponding wild-type protein, such as an activity of at least 2, alternatively at least 5, alternatively at least 10, alternatively at least 20, alternatively at least 25, alternatively at least 50, alternatively at least 100, alternatively at least 250, alternatively at least 500, alternatively at least 1000, alternatively at least 2000 times higher than the corresponding wild-type protein (i.e., when assessed under the same conditions in accordance with the method).

The cytochrome P450 variant may be utilized in the method in any form. For example, in certain embodiments, the cytochrome P450 variant is utilized as a whole cell catalyst, i.e., a composition comprising host cells expressing the cytochrome P450 variant. Examples of such host cells, as well as various techniques for preparing such whole cell catalysts comprising the cytochrome P450 variant are described above. As such, in some embodiments, the method comprises preparing a whole cell catalyst comprising (e.g. expressing) the cytochrome P450 variant, and subsequently combining the whole cell catalyst with the organosilicon compound to prepare the organosilicon compound having at least one silicon-bonded carbinol group and/or at least one silanol group. In particular embodiments, the cytochrome P450 variant is utilized as a cell lysate, i.e., a composition comprising a lysis product of the whole cell catalyst comprising the cytochrome P450 variant described above. In yet other embodiments, the cytochrome P450 variant is utilized as an isolated enzyme. For example, in such embodiments, the method comprises isolating and/or purifying the cytochrome P450 variant from the host cells expressing the cytochrome P450 variant and/or the cell lysate described above to give an isolated cytochrome P450 variant, which is then combined with the organosilicon compound to prepare the organosilicon compound having at least one silicon-bonded carbinol group and/or at least one silanol group.

As introduced above, the method of preparing the organosilicon compound having at least one silicon-bonded carbinol group and/or at least one silanol group comprises preparing a reaction mixture by combining the cytochrome P450 variant, an initial organosilicon compound and a cofactor. More specifically, the initial organosilicon compound comprises at least one silicon-bonded hydrocarbyl group capable of being oxidized to a silicon-bonded carbinol group or a silanol group (i.e., a silicon-bonded hydroxyl group) when the reaction mixture is exposed to an oxidant. The silicon-bonded hydrocarbyl group is unsubstituted and free from heteroatoms.

The initial organosilicon compound may vary widely with respect to the other substituents bonded to silicon. Each silicon-bonded hydrocarbyl group present in the initial organosilicon compound is independently selected. For clarity and consistency, reference below to “the silicon-bonded hydrocarbyl group” or “the hydrocarbyl group” refers to the at least one hydrocarbyl group of the initial organosilicon compound, but can also apply to other silicon-bonded hydrocarbyl groups present in the initial organosilicon compound, whether or not oxidized via the method, which should not be construed to mean that each hydrocarbyl group is identical.

Hydrocarbyl groups suitable for inclusion in the initial organosilicon compound and for oxidation via the method include monovalent hydrocarbon moieties, as well as derivatives and modifications thereof, which may independently be linear, branched, cyclic, or combinations thereof, and saturated or unsaturated. However, the hydrocarbyl group of the initial organosilicon compound that is oxidized via the method is unsubstituted. With regard to such hydrocarbyl groups, the term “unsubstituted” describes hydrocarbon moieties composed of carbon and hydrogen atoms, i.e., without heteroatom substituents. The term “substituted” describes hydrocarbon moieties where either at least one hydrogen atom is replaced with an atom or group other than hydrogen (e.g. a halogen atom, an alkoxy group, an amine group, etc.) (i.e., as a pendant or terminal substituent), a carbon atom within a chain/backbone of the hydrocarbon is replaced with an atom other than carbon (e.g. a heteroatom, such as oxygen, sulfur, nitrogen, etc.) (i.e., as a part of the chain/backbone), or both.

Linear and branched hydrocarbyl groups may independently be saturated or unsaturated and, when unsaturated, may be conjugated or nonconjugated. Cyclic hydrocarbyl groups may independently be monocyclic or polycyclic, and encompass cycloalkyl groups, aryl groups, and heterocycles, which may be aromatic, saturated and nonaromatic and/or non-conjugated, etc. Examples of combinations of linear and cyclic hydrocarbyl groups include alkaryl groups, aralkyl groups, etc. General examples of hydrocarbon moieties suitably for use in or as the hydrocarbyl group include alkyl groups, aryl groups, alkenyl groups, alkynyl groups, and the like, as well as derivatives, modifications, and combinations thereof. Examples of alkyl groups include methyl, ethyl, propyl (e.g. iso-propyl and/or n-propyl), butyl (e.g. isobutyl, n-butyl, tert-butyl, and/or sec-butyl), pentyl (e.g. isopentyl, neopentyl, and/or tert-pentyl), hexyl, and the like (i.e., other linear or branched saturated hydrocarbon groups, e.g. having greater than 6 carbon atoms). Examples of aryl groups include phenyl, tolyl, xylyl, naphthyl, benzyl, dimethyl phenyl, and the like, as well as derivatives and modifications thereof, which may overlap with alkaryl groups (e.g. benzyl) and aralkyl groups (e.g. tolyl, dimethyl phenyl, etc.). Examples of alkenyl groups include vinyl, allyl, propenyl, isopropenyl, butenyl, isobutenyl, pentenyl, heptenyl, hexenyl, cyclohexenyl groups, and the like, as well as derivatives and modifications thereof.

In certain embodiments, the silicon-bonded hydrocarbyl group of the initial organosilicon compound is an unsubstituted hydrocarbyl group having from 1 to 12 carbon atoms. For example, in some such embodiments, the hydrocarbyl group is an alkyl group, such as an alkyl group having from 1 to 6, alternatively from 1 to 5, alternatively from 1 to 4 carbon atoms. Specific examples of alkyl groups include methyl groups, ethyl groups, propyl groups (e.g. n-propyl and iso-propyl groups), butyl groups (e.g. n-butyl, sec-butyl, iso-butyl, and tert-butyl groups), pentyl groups, hexyl groups, heptyl groups, etc., and the like, as well as derivatives and/or modifications thereof. In other embodiments, the hydrocarbyl group is an unsubstituted alkenyl groups having from 2 to 6 carbon atoms, such as from 2 to 5, alternatively from 2 to 4, alternatively from 2 to 3 carbon atoms.

In particular embodiments, the hydrocarbyl group is selected from substituted and unsubstituted aryl, alkaryl, and aralkyl groups having from 1 to 12 carbon atoms, such as from 2 to 12, alternatively from 2 to 10, alternatively from 3 to 10, alternatively from 3 to 8, alternatively from 4 to 8 carbon atoms. In particular embodiments, the at least one R is independently selected from unsubstituted aryl, alkaryl, and aralkyl groups.

In certain embodiments, the initial organosilicon compound may also include substituted hydrocarbyl groups that are not oxidized via the inventive method. For example, the initial organosilicon compound may include one or more halocarbon groups.

General examples of halocarbon groups include halogenated derivatives of the hydrocarbon moieties above, such as halogenated alkyl groups (e.g. any of the alkyl groups described above, where one or more hydrogen atoms is replaced with a halogen atom such as F or Cl), aryl groups (e.g. any of the aryl groups described above, where one or more hydrogen atoms is replaced with a halogen atom such as F or Cl), and combinations thereof. Examples of halogenated alkyl groups include fluoromethyl, 2-fluoropropyl, 3,3,3-trifluoropropyl, 4,4,4-trifluorobutyl, 4,4,4,3,3-pentafluorobutyl, 5,5,5,4,4,3,3-heptafluoropentyl, 6,6,6,5,5,4,4,3,3-nonafluorohexyl, and 8,8,8,7,7-pentafluorooctyl, 2,2-difluorocyclopropyl, 2,3-difluorocyclobutyl, 3,4-difluorocyclohexyl, 3,4-difluoro-5-methylcycloheptyl, chloromethyl, chloropropyl, 2-dichlorocyclopropyl, 2,3-dichlorocyclopentyl, and the like, as well as derivatives and modifications thereof. Examples of halogenated aryl groups include chlorobenzyl, pentafluorophenyl, fluorobenzyl groups, and the like, as well as derivatives and modifications thereof.

In specific embodiments, the initial organosilicon compound includes only silicon-bonded unsubstituted hydrocarbyl groups selected from those described above, and is free from any silicon-bonded substituted hydrocarbyl groups.

The initial organosilicon compound is not limited and may comprise any combination of siloxy groups. Further still, the initial organosilicon compound may be free from siloxy groups, which are typically only present in organosilicon compounds including one or more siloxane bonds, which may alternatively be referred to as organosiloxanes or polyorganosiloxanes.

Examples of suitable siloxy groups include [M], [D], [T], and [Q] units/siloxy groups, which, as understood in the art, each represent structural units of individual functionality present in siloxanes, such as organosiloxanes and organopolysiloxanes. More specifically, [M] represents a monofunctional unit of general formula R″₃SiO_{1/2}; [D] represents a difunctional unit of general formula R″₂SiO_{2/2}; [T] represents a trifunctional unit of general formula R″SiO_{3/2}; and [Q] represents a tetrafunctional unit of general formula SiO_{4/2}, as shown by the general structural moieties below:

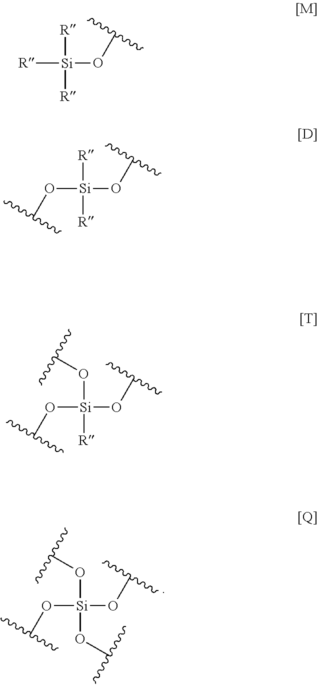

In these general structural moieties, each R″ is independently a monovalent substituent, such as the silicon-bonded hydrocarbyl group of the initial silicon compound described above.

In certain embodiments, the initial organosilicon compound is free from T and/or Q siloxy units. In these or other embodiments, the initial organosilicon compound consists of only M and/or D siloxy units. Alternatively, the initial organosilicon compound can be an organosilane or an organosiloxane. The organosilane can be a monosilane, disilane, trisilane, or polysilane. Similarly, the organosiloxane can be a disiloxane, trisiloxane, or polysiloxane.

In various embodiments, the initial organosilicon compound has the general formula (I) or (II):

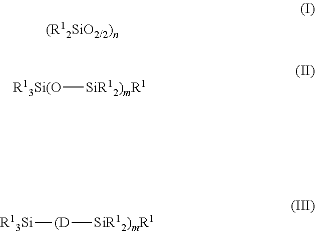

where each R¹ is an independently selected unsubstituted hydrocarbyl group or H, with the proviso that at least one of R¹ is a hydrocarbyl group, subscript n is from 3 to 8, subscript m is from 0 to 15, and each D is an independently selected divalent linking group.

In a specific embodiment, the initial organosilicon compound has the general formula (I) above. As understood in the art, when the initial organosilicon compound has the general formula (I), the initial organosilicon compound is a cyclic siloxane. Because subscript n is from 3 to 8, the cyclic siloxane has from 3 to 8 D siloxy units.

Examples of cyclic siloxanes include hexamethyl cyclotrisiloxane (D3), octamethyl cyclotetrasiloxane (D4), decamethyl cyclopentasiloxane (D5), dodecamethyl-cyclohexasiloxane (D6), 1,1-diethylhexamethyl cyclotetrasiloxane, phenylheptamethyl cyclotetrasiloxane, 1,1-diphenylhexamethyl cyclotetrasiloxane, 1,3,5,7-tetravinyltetramethyl cyclotetrasiloxane, 1,3,5,7-tetramethyl cyclotetrasiloxane, 1,3,5,7-tetracyclohexyltetramethyl cyclotetrasiloxane, tris(3,3,3-trifluoropropyl)trimethylcyclotrisiloxane, 1,3,5,7-tetra(p-vinylphenyl) tetramethyl cyclotetrasiloxane, 1,3,5,7-tetra[3-(p-vinylphenyl) propyl]tetramethyl cyclotetrasiloxane, and the like. Furthermore, a mixture of different cyclic siloxanes may be utilized.

In another specific embodiment, the initial organosilicon compound has the general formula (II) above. When subscript m is 0, the initial organosilicon compound of general formula (II) is a silane compound. When subscript m is 1, the initial organosilicon compound of general formula (II) is a disiloxane compound. When subscript m is 2, the initial organosilicon compound of general formula (II) is a trisiloxane compound, and so on.

When the initial organosilicon compound has the general formula (II) above and subscript m is 0, specific examples of the organosilicon compound include, but are not limited to, silanes having the following formulae: Me₄Si, Et₄Si, Me₃EtSi, Me₂Ph₂Si, Vi₄Si, PhSiVi₃, MeSiVi₃, PhMeSiVi₂, Ph₂SiVi₂, and PhSi(CH₂CH═CH₂)₃, wherein Me is methyl, Eth is ethyl, Ph is phenyl, and Vi is vinyl.

When the initial organosilicon compound has the general formula (II) above and subscript m is 1, and each R¹ is methyl, the initial organosilicon compound is hexamethyldisiloxane. On of skill in the art readily appreciates other species of siloxane compounds within the scope of general formula (II) above based on the selection of each R¹ and subscript m.

In another specific embodiment, the initial organosilicon compound has the general formula (III) above. The initial organosilicon compound of general formula (III) is distinct from that based on general formula (II) based on the selection of D, the divalent linking group. For example, if D were oxygen (—O—), then the initial organosilicon compound of general formula (III) is the same as that of general formula (II) when, in general formula (II), subscript m is 1 and each R¹ is the same between general formulas (II) and (III). However, the divalent linking group D is typically a hydrocarbon group. In specific embodiments, the divalent linking group D is a hydrocarbon group free from heteroatoms. In further specific embodiments, the divalent linking group D is unsubstituted.

Suitable hydrocarbon groups are described above with regard to the hydrocarbyl group of the initial organosilicon compound, with the exception being that the hydrocarbyl group of the initial organosilicon compound is monovalent and the linking group D is divalent. Thus, as readily understood in the art, a hydrogen atom is removed from any of the hydrocarbyl groups described above if utilized as the divalent linking group D. For example, the hydrocarbyl group of the initial organosilicon compound can be methyl (—CH₃), whereas the divalent linking group D of the initial organosilicon compound be methylene (—CH₂—). The divalent linking group can also be branched. By way of one specific example, when the divalent linking group is —C₂H₄—, it may be represented by —CH₂CH₂—, which is linear, or CH(CH₃), which is branched. One of skill in the art readily appreciates other linear and branched structures suitable for the divalent linking group D. Further, the divalent linking group can comprise or consist of an arylene group, for example, in lieu of a linear hydrocarbon group or a saturated hydrocarbon group. In certain embodiments, the divalent linking group D has from 1 to 10, alternatively from 1 to 9, alternatively from 1 to 8, alternatively from 1 to 7, alternatively from 1 to 6, alternately from 1 to 5, alternatively from 1 to 4, alternatively of m1 to 3, alternatively 1 or 2, carbon atoms.

In certain embodiments, the initial organosilicon compound has the general formula (III), and subscript m is 0. In these embodiments, the initial organosilicon compound has the general formula R¹₃Si-D-SiR¹₃. When D is methylene, for example, the initial organosilicon compound has the general formula R¹₃Si—CH₂—SiR¹₃.

In specific embodiments, the initial organosilicon compound may be a silicone fluid. Specific examples of suitable silicone fluids include hexamethylcyclotrisiloxane, octamethylcyclotetrasiloxane, decamethylcyclopentasiloxane, dodecamethylcyclohexasiloxane, octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, hexamethyl-3,3, bis{(trimethylsilyl)oxy}trisiloxane pentamethyl{(trimethylsilyl)oxy}cyclotrisiloxane as well as polydimethylsiloxanes, polyethylsiloxanes, polymethylethylsiloxanes, polymethylphenylsiloxanes, polydiphenylsiloxanes, caprylyl methicone, hexamethyldisiloxane, heptamethyloctyltrisiloxane, hexyltrimethicone, and the like, as well as derivatives, modifications, and combinations thereof.

The initial organosilicon compound may be prepared or otherwise obtained, i.e., as a prepared compound. Methods of preparing the initial organosilicon compound are known in the art, with such compounds and suitable starting materials commercially available from various suppliers. Preparing the initial organosilicon compound when part of the method is typically performed prior to combining the same with the cytochrome P450 variant.

Likewise, the initial organosilicon compound may be utilized in any form, such as neat (i.e., absent solvents, carrier vehicles, diluents, etc.), or disposed in a carrier vehicle, such as a solvent or dispersant. For example, the initial organosilicon compound may be disposed in a carrier vehicle, such as one of those described herein. It will be appreciated that the acryloxy-functional organosilicon monomer may be combined with the carrier vehicle, if utilized, prior to, during, or after being combined with the cytochrome P450 variant. In some embodiments, the initial organosilicon compound is utilized free from, alternatively substantially free from carrier vehicles. For example, in certain embodiments, the method may comprise stripping the initial organosilicon compound of volatiles and/or solvents, or distilling the initial organosilicon compound from solvents, volatiles, etc., to prepare the initial organosilicon compound for use in the method.

The initial organosilicon compound may comprise but one type of organosilicon compound or, alternatively, may comprise more than one type of organosilicon compound, such as two, three, or more organosilicon compounds that differ from one another with regard to structure, viscosity, the hydrocarbyl group, etc.

The initial organosilicon compound may be utilized in any amount, which will be selected by one of skill in the art, e.g. dependent upon the particular components selected for reacting, the reaction parameters employed, the scale of the reaction (e.g. total amounts of the initial organosilicon compound to be reacted and/or organosilicon compound having at least one silicon-bonded carbinol group and/or at least one silanol group to be prepared), etc.

The method of preparing the organosilicon compound having at least one silicon-bonded carbinol group and/or at least one silanol group comprises combining the cytochrome P450 variant and the initial organosilicon compound in the presence of an oxidant and a cofactor and, optionally, any other components utilized (collectively, the “reaction components”). As will be understood by those of skill in the art, there is generally no proactive step required for the cytochrome P450 variant-facilitated reaction of the initial organosilicon compound and the oxidant beyond combining the components together. As introduced above, the reaction of the method may be generally defined or otherwise characterized as an oxidation and/or hydroxylation reaction, and certain parameters and conditions of the reaction may be selected by those known in the art of such reactions in order to prepare the organosilicon compound having at least one silicon-bonded carbinol group and/or at least one silanol group.

Typically, the reaction components are reacted in a vessel or reactor to prepare the organosilicon compound having at least one silicon-bonded carbinol group and/or at least one silanol group. More specifically, the reaction components are typically combined in the vessel to prepare a reaction mixture, such that the organosilicon compound having at least one silicon-bonded carbinol group and/or at least one silanol group is prepared from the reaction mixture.

As introduced above, the reaction mixture may comprise components other than the cytochrome P450 variant, the initial organosilicon compound, and the oxidant. Typically, the oxidant is oxygen in atmospheric air, which does not require any proactive steps of incorporation of an oxidant due to its inherent availability in ambient conditions. Alternatively, the oxidant may be peroxide, which may be any peroxide source. For example, the cytochrome P450 variant and the initial organosilicon compound are typically combined in the presence of a carrier vehicle (e.g. a solvent, diluent, fluid, etc., or a combination thereof), such that the reaction mixture comprises a solution, emulsion, suspension, slurry, biphasic mixture, or combinations thereof. The particular solvents, carriers, and/or diluents utilized, and the respective amounts thereof employed, will be independently selected by one of skill in the art, e.g. based the particular reaction components being utilized, the particular initial organosilicon compound having at least one silicon-bonded carbinol group and/or at least one silanol group being prepared, the scale of the reaction, etc. For example, it is understood by those of skill in the art that biocatalytic reactions may be conducted heterogeneously, e.g. with one or more components suspended, but not dissolved, in the carrier vehicle. Typically, however, certain reaction components are employed as homogeneous mixtures (i.e., prior to forming the reaction mixture) and/or the reaction mixture itself is substantially homogeneous. In general, solvents, carriers, and/or diluents utilized will be selected to help fluidize and/or compatibilize one or more of the reaction components, without promoting undesired reactions of the reaction components. As such, examples of particular carrier vehicles include solvents, fluids, etc. suitable to sufficiently carry, dissolve, and/or disperse any component(s) of the reaction mixture during the preparation of the organosilicon compound having at least one silicon-bonded carbinol group and/or at least one silanol group.

Examples of suitable solvents include aqueous solvents (e.g. water and water miscible organic solvents), organic solvents, fluids, oil (e.g. an organic oil and/or a silicone oil), etc., as well as combinations thereof. Typically, the carrier vehicle comprises an aqueous solvent comprising, alternatively consisting essentially of, water. However, in certain embodiments, additional and/or alternative carrier fluids and/or diluents may also be utilized, such as any of those described herein. For example, in some embodiments, the carrier vehicle comprises an organic solvent. Examples of organic solvents include those comprising an alcohol, such as methanol, ethanol, isopropanol, butanol, and n-propanol; a ketone, such as acetone, methylethyl ketone, and methyl isobutyl ketone; an aromatic hydrocarbon, such as benzene, toluene, and xylene; an aliphatic hydrocarbon, such as heptane, hexane, and octane; a glycol ether, such as propylene glycol methyl ether, dipropylene glycol methyl ether, propylene glycol n-butyl ether, propylene glycol n-propyl ether, and ethylene glycol n-butyl ether; an acetate, such as ethyl acetate, butyl acetate, ethylene glycol monoethyl ether acetate, and propylene glycol methyl ether acetate; a halogenated hydrocarbon, such as dichloromethane, 1,1,1-trichloroethane, and chloroform; dimethyl sulfoxide; dimethyl formamide, acetonitrile; tetrahydrofuran; white spirits; mineral spirits; naphtha; n-methylpyrrolidone; and the like, as well as derivatives, modifications, and combination thereof. In certain embodiments, the carrier vehicle comprises a polar organic solvent, such as a solvent compatible with water. Specific examples of such polar organic solvents include methanol, ethanol, 1-propanol, 2-propanol, 2-methyl-2-propanol, 2-butanone, tetrahydrofuran, acetone, and combinations thereof.

In certain embodiments, the carrier vehicle comprises an organic fluid, which typically comprises an organic oil including a volatile and/or semi-volatile hydrocarbon, ester, and/or ether. General examples of such organic fluids include volatile hydrocarbon oils, such as C₆-C₁₆ alkanes, C₈-C₁₆ isoalkanes (e.g. isodecane, isododecane, isohexadecane, etc.), C₈-C₁₆ branched esters (e.g. isohexyl neopentanoate, isodecyl neopentanoate, etc.), and the like, as well as derivatives, modifications, and combinations thereof. Additional examples of suitable organic fluids include aromatic hydrocarbons, aliphatic hydrocarbons, alcohols having more than 3 carbon atoms, aldehydes, ketones, amines, esters, ethers, glycols, glycol ethers, acetates, alkyl halides, aromatic halides, and combinations thereof. Hydrocarbons include isododecane, isohexadecane, Isopar L (C₁₁-C₁₃), Isopar H (C₁₁-C₁₂), hydrogenated polydecene. Ethers and esters include isodecyl neopentanoate, neopentylglycol heptanoate, glycol distearate, dicaprylyl carbonate, diethylhexyl carbonate, propylene glycol n-butyl ether, ethyl-3 ethoxypropionate, propylene glycol methyl ether acetate, tridecyl neopentanoate, propylene glycol methylether acetate (PGMEA), propylene glycol methylether (PGME), octyldodecyl neopentanoate, diisobutyl adipate, diisopropyl adipate, propylene glycol dicaprylate/dicaprate, octyl ether, octyl palmitate, and combinations thereof.

In some embodiments, the carrier vehicle comprises a silicone fluid. The silicone fluid is typically a low viscosity and/or volatile siloxane. If utilized as a carrier vehicle, the silicone fluid is different from the initial organosilicon compound utilized in the method, or the initial organosilicon compound itself may serve as a carrier fluid in the reaction mixture.

Other carrier vehicles may also be utilized. For example, in some embodiments, the carrier vehicle comprises an ionic liquid. Examples of ionic liquids include anion-cation combinations. Generally, the anion is selected from alkyl sulfate-based anions, tosylate anions, sulfonate-based anions, bis(trifluoromethanesulfonyl)imide anions, bis(fluorosulfonyl)imide anions, hexafluorophosphate anions, tetrafluoroborate anions, and the like, and the cation is selected from imidazolium-based cations, pyrrolidinium-based cations, pyridinium-based cations, lithium cation, and the like. However, combinations of multiple cations and anions may also be utilized. Specific examples of the ionic liquids typically include 1-butyl-1-methylpyrrolidinium bis(trifluoromethanesulfonyl)imide, 1-methyl-1-propylpyrrolidinium bis-(trifluoromethanesulfonyl)imide, 3-methyl-1-propylpyridinium bis(trifluoromethanesulfonyl)imide, N-butyl-3-methylpyridinium bis(trifluoromethanesulfonyl)imide, 1-methyl-1-propylpyridinium bis(trifluoromethanesulfonyl)imide, diallyldimethylammonium bis(trifluoromethanesulfonyl)imide, methyltrioctylammonium bis(trifluoromethanesulfonyl)imide, 1-butyl-3-methylimidazolium bis(trifluoromethanesulfonyl)imide, 1,2-dimethyl-3-propylimidazolium bis(trifluoromethanesulfonyl)imide, 1-ethyl-3-methylimidazolium bis(trifluoromethanesulfonyl)imide, 1-vinylimidazolium.bis(trifluoromethanesulfonyl)imide, 1-allyl imidazolium bis(trifluoromethanesulfonyl)imide, 1-allyl-3-methylimidazolium bis(trifluoromethanesulfonyl)imide, lithium bis(trifluoromethanesulfonyl)imide, and the like, as well as derivatives, modifications, and combinations thereof.

The carrier vehicle may comprise a combination of different vehicles/solvents/diluents, etc., which may be miscible or immiscible with one another. For example, as introduced above, the reaction mixture may be homogenous or heterogeneous (e.g. in the form of an emulsion, such as a water-in-oil emulsion, silicone-in-oil emulsion, oil-in-water emulsion, oil-in-silicone emulsion, etc.

In certain embodiments, the reaction mixture may comprise one or more additional components, which will be selected by those of skill in the art in view of the particular parameters employed in the method. Examples of such additional components include buffers (e.g. M9-N buffer, 2-(N-morpholino) ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline solutions, sodium citrate, sodium acetate, sodium borate, etc.), reducing agents and/or cofactors (e.g. NADPH, NADP+ (with a cofactor regeneration system), NADH, sodium dithionite, dithiothreitol (DTT), β-mercaptoethanol (BME), tris(2-carboxyethyl)phosphine (TCEP), etc.), chelators (e.g. 2-({2-[bis(carboxymethyl)amino]ethyl} (carboxymethyl)amino)acetic acid (EDTA), ethylene glycol-bis(2-aminoethylether)-N,N,N′,N′-tetraacetic acid (EGTA), 1,2-bis(o-aminophenoxy) ethane-N,N,N′,N′-tetraacetic acid (BAPTA), etc.), salts (e.g. halide salts of sodium, calcium, potassium, magnesium, etc., such as NaCl, KCl, CaCl₂, etc.), cosolvents and/or diluents (e.g. dimethylsulfoxide, dimethylformamide, ethanol, methanol, isopropanol, glycerol, tetrahydrofuran, acetone, acetonitrile, acetic acid, etc.), denaturants (e.g. urea, guandinium hydrochloride, etc.), detergents (sodium dodecylsulfate and Triton-X 100) and/or surfactants (e.g. cationic, anionic, nonionic, and/or zwitterionic surfactants), sugars (e.g. glucose, sucrose, etc.), as well as combinations thereof.

When utilized, such additional components can be used in any amount, loading, and/or to suitable concentration, which can be readily determined by one of skill in the art. In general, such components are typically present in the reaction mixture, when utilized, at concentrations of from 1 μM to 1 M, such as in concentration of about 1, 10, or 100 μM, about 1, 10, 25, 50, 100, 250, or 500 mM, or about 1 M. In some embodiments, a reducing agent is used in a substoichiometric amount with respect to the initial organosilicon compound. Cosolvents, when utilized, may be pressing in the reaction mixture in an amount of from 1 to 75% (v/v), such from 1 to 50, alternatively from 1 to 25, alternatively from 1 to 10, alternatively from 1 to 5%.

As introduced above, the cytochrome P450 variant may be utilized in the method in any form, such as in the form of a whole cell catalyst, a cell lysate, or a protein isolate. Alternatively, the cytochrome P450 variant may be utilized in the form of a lyophilized cell lysate, which can be hydrated with deionized water to yield a reconstituted lysate. As such, it will be appreciated that the reaction mixture may comprise a suspension of such cells and/or cellular components. For example, the reaction may be conducted in vivo with intact cells expressing cytochrome P450 variant such that, in some embodiments, the method comprises preparing a suspension of the whole cell catalyst in a suitable medium supplemented with nutrients (e.g. mineral micronutrients, glucose and other fuel sources, cofactors, if necessary for the reaction, etc.). As will be understood by those of skill in the art, yields of the organosilicon compound having at least one silicon-bonded carbinol group and/or at least one silanol group may be controlled, in part, by selecting the cell density in the reaction mixtures. For example, in some embodiments, the reaction mixture comprises a cellular suspension exhibiting optical densities in the range of from 0.1 to about 50 at 600 nm may be employed. However, it will be appreciated that densities outside this range may also be utilized, e.g. depending on the type of host cell utilized, the specific cytochrome P450 variant being expressed, etc.

In certain embodiments, the reaction mixture is pH adjusted and/or controlled. The pH may be monitored, adjusted, controlled, etc. by any method known in the art, with the pH adjusting generally comprising adding an acid (e.g. HCl), a base (e.g. NaOH), a buffer, or combinations thereof, to the reaction mixture itself (i.e., once formed) or to one or more of the reaction components. For example, in certain embodiments, the cytochrome P450 variant is utilized in a pH adjusted and/or controlled composition prior to being combined with the initial organosilicon compound. In general, the reaction is carried out at a pH of from 7 to about 9, alternatively of about 8. For example, in certain embodiments, the reaction mixture is formulated to comprise a pH (e.g. upon formation and/or during the reaction) of from 7.5 to 8.5, such as from 7.6 to 8.4, alternatively from 7.7 to 8.3, alternatively from 7.8 to 8.2, alternatively from 7.9 to 8.1. In particular embodiments, the pH of the reaction mixture is adjusted to and/or maintained at a pH of about 8 during the reaction. It is to be appreciated that values outside of these ranges may also be utilized, as will be understood by those of skill in the art in view of the description herein. For example, in particular embodiments, the particular pH of the reaction mixture will typically be adjusted to optimize the activity of the particular cytochrome P450 variant utilized, and may also be varied during the method (e.g. in real-time) to increase/decrease the rate of the oxidation reaction and/or to stop the reaction altogether.

The reaction components can be utilized in varying amounts and/or ratios, which will be selected by those of skill in the art.

Typically, the cytochrome P450 variant is utilized in a catalytic amount, i.e., a substoichiometric amount with respect to the initial organosilicon compound. For example, in certain embodiments, the cytochrome P450 variant is utilized in an amount of from 0.001 to 10 mol %, such as from 0.001 to 5, alternatively from 0.001 to 1, alternatively from 0.001 to 0.5, alternatively from 0.001 to 0.2, alternatively from 0.01 to 0.2 mol %. In these or other embodiments, the cytochrome P450 variant may be utilized in an amount sufficient to provide the reaction mixture with a concentration of the cytochrome P450 variant of at least 0.1 μM, such as a concentration of from 0.1 to 10, alternatively from 0.1 to 5 μM, alternatively from 0.1 to 1 μM. In some embodiments, the cytochrome P450 variant is utilized in an amount sufficient to provide the reaction mixture with a concentration of the cytochrome P450 variant of from 1 to 15, such as from 1 to 10 μM.

The amount of the initial organosilicon compound utilized in the method is not limited, and will be selected in view of the size/scale of the reaction, the particular species, properties, and loading of the cytochrome P450 variant utilized, etc. In general, the initial organosilicon compound is utilized in an amount sufficient to provide the reaction mixture with a concentration of the initial organosilicon compound of at least 1 mM, such as such as a concentration of from 1 to 50, alternatively from 1 to 25, alternatively from 1 to 15, alternatively from 5 to 15, alternatively from 5 to 10 mM. However, concentrations outside these ranges may also be utilized, and one of skill in the art will select the particular amounts of the initial organosilicon compound in view of the reaction parameters employed. For example, in some embodiments, the initial organosilicon compound utilized, e.g. in view of the particular silicone-acrylate polymer being prepared, the particular monomers utilized, etc.

The method may utilize any conditions suitable for promoting the catalytic oxidation of the initial organosilicon compound in the reaction mixture, and any technique, equipment, or procedure known in the art for achieving such conditions. For example, when the reaction is carried out at an elevated temperature as described below, the vessel or reactor may be heated or cooled in any suitable manner, e.g. via a jacket, mantle, exchanger, bath, coils, etc. Likewise, the method may comprise agitating the reaction mixture during and/or after formation. The agitating may enhance mixing and contacting together the reaction components when combined, e.g. in the reaction mixture. The method may also include independently employing other conditions tailored to enhance the contacting with (e.g. concurrently or sequentially) or without (i.e., independent from, alternatively in place of) the agitating. These or other conditions may be result-effective conditions for enhancing reaction yield of the organosilicon compound having at least one silicon-bonded carbinol group and/or at least one silanol group. Conditions may independently be an ambient condition (e.g. room temperature and/or atmospheric pressure) and/or a non-ambient parameter (e.g. reduced or elevated temperature and/or reduced or elevated pressure). Additionally, reaction parameters may be dynamically modified, modified in real time (i.e., during the reaction), or may be static (e.g. for the duration of the reaction, or for any portion thereof). For example, temperature, pH, agitation, oxygen content, etc., as well as other parameters, may be independently selected or modified during the reaction.

The reaction is typically conducted under aerobic conditions, as an oxidant, such as oxygen, is a necessary component of the oxidation reaction. However, the reactions can be conducted under an inert atmosphere, such as a nitrogen atmosphere, argon atmosphere, etc., so long as an oxidant is introduced to or otherwise present in the reaction mixture. For example, oxidant may be introduced to or otherwise combined with the reaction mixture via exposure of the reaction mixture to ambient atmosphere, via oxygen bubbler, etc. Alternatively, the oxidant may be peroxide, and/or abiotic oxidants may be utilized. As such, it will be appreciated the reaction mixture is typically prepared in the presence of oxygen, but may be prepared under anaerobic conditions and subsequently combined and/or exposed to oxygen.

The reaction may be carried out at any temperature compatible with the cytochrome P450 variant. In general, the reaction is carried out at a temperature of from 4 to 45° C. In some embodiments, the reaction is carried out at an elevated temperature. The elevated temperature will be selected and controlled depending on the particular reaction components selected, such as whether the cytochrome P450 variant is provided as an isolated enzyme or in the form of the whole-cell catalyst. Accordingly, the elevated temperature will be readily selected by one of skill in the art in view of the reaction conditions and parameters selected and the description herein. The elevated temperature is typically from greater than 25° C. (ambient temperature) to 45° C., such as from 30 to 45, alternatively from 30 to 40, alternatively from 35 to 40° C.

The time during which the reaction to prepare the organosilicon compound having at least one silicon-bonded carbinol group and/or at least one silanol group is carried out is a function of scale, reaction parameters and conditions utilized, the reaction components selected, etc. In certain embodiments, the reaction may be carried out for a duration of ranging from a few minutes to many hours, such as from 5 minutes to 72 hours. However, the components and conditions of the hydrolysis reaction are typically selected to facilitate hydrolysis during a duration of from 30 minutes to 48 hours, such as from 1 to 48, alternatively from 4 to 48, alternatively from 4 to 24 hours. However, it is to be appreciated that durations outside of these ranges/values may also be utilized, as will be understood by those of skill in the art in view of the description herein. For example, on a relatively large scale (e.g. a scale of greater than 1, alternatively greater than 5, alternatively greater than 10, alternatively greater than 50, alternatively greater than 100 kg), or with a difficult substrate, the reaction may be carried out for one or more days, such as for at least 1, alternatively at least 2, alternatively at least 3, alternatively at least 5 days.

Generally, the reaction of the components of the reaction mixture prepares a reaction product comprising the organosilicon compound having at least one silicon-bonded carbinol group and/or at least one silanol group. In particular, over the course of the reaction, the reaction mixture comprises increasing amounts of the organosilicon compound having at least one silicon-bonded carbinol group and/or at least one silanol group being prepared and decreasing amounts of the initial organosilicon compound utilized in the reaction. Once the reaction is complete (e.g. the initial organosilicon compound is consumed, no additional amount of organosilicon compound having at least one silicon-bonded carbinol group and/or at least one silanol group is being prepared, etc.), the reaction mixture may be referred to as the reaction product comprising the organosilicon compound having at least one silicon-bonded carbinol group and/or at least one silanol group. In this fashion, the reaction product typically includes any remaining amounts of the reaction components, as well as degradation and/or reaction products thereof. For example, when the reaction is carried out in the carrier vehicle, the reaction product will include the solvents/fluids thereof.

Accordingly, in certain embodiments, the method further comprises isolating the organosilicon compound having at least one silicon-bonded carbinol group and/or at least one silanol group from the reaction product. As used herein with regard to the organosilicon compound having at least one silicon-bonded carbinol group and/or at least one silanol group of the reaction product, the term “isolating” refers to a process of increasing the relative concentration of the organosilicon compound having at least one silicon-bonded carbinol group and/or at least one silanol group as compared to other compounds in combination therewith (e.g. in the reaction product or a purified version thereof). As such, as is understood in the art, isolating may comprise removing the other compounds from such a combination (i.e., decreasing the amount of impurities combined with the organosilicon compound having at least one silicon-bonded carbinol group and/or at least one silanol group, e.g. in the reaction product) and/or removing the organosilicon compound having at least one silicon-bonded carbinol group and/or at least one silanol group itself from the combination. Any suitable technique and/or protocol for isolation may be utilized. Examples of suitable isolation techniques include centrifugation, distillation, concentration/stripping/evaporation, washing and/or extraction, filtration, partitioning/phase separation (e.g. based on solubility, freeze point, etc.), chromatographic methods (e.g. column chromatography, size exclusion chromatography, ion exchange chromatography, affinity chromatography, hydrophobic interaction chromatography, reverse phase chromatography, etc.), and the like. As will be understood by those of skill in the art, any of these techniques may be used in combination (i.e., sequentially) with any other technique to isolate the organosilicon compound having at least one silicon-bonded carbinol group and/or at least one silanol group.

It is to be appreciated that isolating may include, and thus may be referred to as, purifying the organosilicon compound having at least one silicon-bonded carbinol group and/or at least one silanol group. However, purifying the organosilicon compound having at least one silicon-bonded carbinol group and/or at least one silanol group may comprise alternative and/or additional techniques as compared to those utilized in isolating the organosilicon compound having at least one silicon-bonded carbinol group and/or at least one silanol group. For example, in certain embodiments where the whole-cell catalyst or cell lysate is employed, general, isolating the organosilicon compound having at least one silicon-bonded carbinol group and/or at least one silanol group may comprise extracting the organosilicon compound having at least one silicon-bonded carbinol group and/or at least one silanol group from the reaction product or removing other components from reaction product (e.g. peptides, biological materials, fats, fibers, oils, carriers, solvents, etc.) to give a crude reaction product comprising the organosilicon compound having at least one silicon-bonded carbinol group and/or at least one silanol group, which is subsequently purified. Regardless of the particular technique(s) selected, isolation and/or purification of organosilicon compound having at least one silicon-bonded carbinol group and/or at least one silanol group may be performed in sequence (i.e., in line) with the reaction itself, and thus may be automated. In other instances, purification may be a stand-alone procedure to which the reaction product comprising the organosilicon compound having at least one silicon-bonded carbinol group and/or at least one silanol group is subjected.

In certain embodiments, as described above, the organosilicon compound having at least one silicon-bonded carbinol group and/or at least one silanol group prepared according to the method is provided as a component of a reaction product, or a purified/isolated form thereof. Such compositions may comprise one or more components in addition to the organosilicon compound having at least one silicon-bonded carbinol group and/or at least one silanol group.

It is to be appreciated that the method described herein is not limited to any particular application, but instead may be utilized in any application involving the oxidation of a suitable initial organosilicon compound to the corresponding organosilicon compound having at least one silicon-bonded carbinol group and/or at least one silanol group. For example, in some embodiments, the method is utilized to prepare the organosilicon compound as a final compound for a desired end use (e.g. as a stand-alone compound, or a component of a functional composition) or as a precursor for use in another reaction (e.g. a condensation or other such reaction for functionalizing and/or derivatizing other organosilicon compounds).

The organosilicon compound having at least one silicon-bonded carbinol group and/or at least one silanol group formed via the method is a function of the initial organosilicon compound utilized. In certain embodiments, at least one silicon-bonded hydrocarbyl group is oxidized and converted to a silanol group in the organosilicon compound prepared in the method. In these or other embodiments, at least one silicon-bonded hydrocarbyl group is oxidized and converted to a silicon-bonded carbinol group in the organosilicon compound prepared in the method. In yet other embodiments, the method oxidizes and converts at least one silicon-bonded hydrocarbyl group of the initial organosilicon compound to a silanol group, and at least one silicon-bonded hydrocarbyl group of the initial organosilicon compound to a carbinol group.

Typically, the silicon-bonded hydrocarbyl group of the initial organosilicon compound and the silicon-bonded carbinol group of the organosilicon compound formed via the method have the same number of carbon atoms. Typically, oxidation and conversion of the silicon-bonded hydrocarbyl group to a silicon-bonded carbinol group involves the replacement of a carbon-hydrogen bond with a C—OH bond. Any carbon atom in the silicon-bonded hydrocarbyl group can be oxidized in the method, and oxidation and conversion is not limited only to a terminal carbon atom in the silicon-bonded hydrocarbyl group of the initial organosilicon compound.

A method of preparing a reaction product is also provided. The method comprises preparing the reaction mixture, exposing the reaction mixture to an oxidant to oxidize and convert the silicon-bonded hydrocarbyl group to the functional group and give an organosilicon compound having the functional group, and reacting the functional group of the organosilicon compound with a group reactive with the functional group of the organosilicon compound, thereby preparing the reaction product.

The functional group of the organosilicon compound is the at least one silanol group and/or silicon-bonded carbinol group. The group reactive with the functional group of the organosilicon compound can be readily determined by one of skill in the art based on the desired reaction product and its end use applications. For example, the functional group of the organosilicon compound may be condensed with another functional group of another organosilicon compound such that the reaction product prepared via the method is a condensation product of the organosilicon compound prepared with the inventive catalyst. For example, in one embodiment, the reaction product is formed via a condensation catalyst between two organosilicon compounds, or an organosilicon compound and another organosilicon compound having a silicon-bonded hydrolysable or hydroxyl group. In these embodiments, the functional group of the organosilicon compound is reacted in the presence of a condensation catalyst.

The condensation catalyst can be any condensation catalyst typically used to promote condensation of silicon-bonded hydroxy (silanol) groups to form Si—O—Si linkages. Examples of condensation catalysts include, but are not limited to, amines, complexes of metals (e.g. lead, tin, zinc, iron, titanium, zirconium) with organic ligands (e.g. carboxyl, hydrocarbyl, alkoxyl, etc.) In particular embodiments, the condensation catalyst can be selected from tin (II) and tin (IV) compounds such as tin dilaurate, tin dioctoate, dibutyltin dilaurate, dibutyltin diacetate, and tetrabutyl tin; and titanium compounds such as titanium tetrabutoxide. In these or other embodiments, the condensation catalyst may be selected from zinc-based, iron-based, and zirconium-based catalysts.

Conventionally, organosilicon compounds having only silicon-bonded hydrocarbyl groups are non-reactive and incredibly stable, allowing for longevity and stability. However, though longevity and stability are desired properties in many end use applications, it's also desirable to degrade, remove, or otherwise convert certain organosilicon compounds. Thus, the inventive method converts initial organosilicon compounds that may be unreactive to organosilicon compounds having a functional group (i.e., a silicon-bonded carbinol group or silanol group), allowing for their further reaction and use. As but one example, the inventive method can be utilized to remove organosilicon compounds from waste streams or other byproduct streams, where the organosilicon compounds would otherwise have to be volatilized or filtered at significant cost.

The following examples, illustrating embodiments of this disclosure, are intended to illustrate and not to limit the invention. Unless otherwise noted, all reactions are carried out under aerobic conditions. Reference to specific mutations in the exemplary cytochrome P450 variants are referred to using conventional residue identification/numbering, disregarding the initial methionine residue (i.e., M1) shown in the sequences of the Sequence Listing (i.e., SEQ ID NOS:1-5).

### Materials

The following examples, illustrating embodiments of this disclosure, are intended to illustrate and not to limit the invention. Unless otherwise noted, all reactions are carried out under aerobic conditions. Reference to specific mutations in the exemplary cytochrome P450 variants are referred to using conventional residue identification/numbering, disregarding the initial methionine residue (i.e., M1) shown in the sequences of the Sequence Listing.

Unless otherwise noted, all other solvents, substrates, and reagents are purchased or otherwise obtained from various commercial suppliers (e.g. Sigma-Aldrich, VWR, Alfa Aesar) and utilized as received (i.e., without further purification).

Phusion polymerase and DpnI are purchased from New England Biolabs (NEB, Ipswich, MA).

Trace Metal Mix used in the protein expression protocols is the trace metal mix set forth in F. W. Studier, Protein production by auto-induction in high density shaking cultures, Protein Expr. Purif. 2005, 41, 207-234, the relevant composition of which is incorporated by reference herein.

Biocatalytic reaction analysis was performed with an Agilent 7820A or an Agilent 8890 gas chromatograph equipped with a 5977B mass spectrometer detector and a DB-5 MS capillary column (30 m length, 0.250 mm diameter, 0.25 μm film thickness using split-mode capillary injection and electron-impact ionization). Calibration curves were generated using serial dilutions of the appropriate material with 5 mM 1,3,5-trimethoxybenzene or 5 mM D4 as an internal standard. Yields were determined by adjusting the calculated concentration of product relative to internal standard, accounting for the dilution factor of enzymatic reaction extraction.

Glucose-6-phosphate dehydrogenase (196 U/mg) was dissolved in a pH 7.4 sodium citrate buffer to give a final concentration of 196 U/mL. The stock of glucose-6-phosphate dehydrogenase was aliquoted in 200 μL portions into individual PCR tubes and flash-frozen in liquid nitrogen (LN₂) or powdered dry ice prior to use. For biocatalytic reactions, a stock of the NADPH cofactor regeneration system was prepared by combining 14 mM NADP⁺, 1.12 M glucose-6-phosphate, and 56 U/mL glucose-6-phosphate dehydrogenase (stock concentrations) in 100 mM pH 7 Tris buffer.

### Cloning, Mutagenesis, and Plasmid Transformation and Isolation

Site-saturation mutagenesis (SSM) experiments were performed using primers bearing degenerate codons (NDT, VHG, TGG) as per the “22-codon trick” known in the art (ACS Synth Biol. 2013, 2, 83-92) and using a modified QuikChange™ protocol. The PCR conditions were as follows: Phusion GC Buffer 1×, 200 mM dNTPs each, 0.5 μM of forward primer, 0.5 μM reverse primer, and 0.02 U/μL of Phusion polymerase. Thermocycling conditions are outlined in Table 1. Upon completion of PCRs, the remaining template was digested with DpnI (1 μL). Gel purification was then performed using gel electrophoresis (1% agarose gel containing a SYBR Gold nucleic acid gel stain) and DNA was visualized on a blue transilluminator. The DNA was then isolated using a Zymoclean DNA gel recovery kit. The purified PCR product was then assembled using the Gibson assembly protocol known in the art. (Nature Methods 2009, 6, 343-345).

TABLE 1
Thermocycling Conditions
Thermocycler Conditions for SSM Protocol

| Step | Temperature (C) | Time (min:sec) | Number of Cycles |
|---|---|---|---|
| Initial | 98 | 00:30 | 1 |
| Denaturation | | | |
| Denaturation | 98 | 00:10 | 5 |
| Annealing | 98 to 55 at | 00:15 | |
| | 1 ºC/sec | | |
| Extension | 72 | 2:06 | |
| Denaturation | 98 | 0:10 | 25 |
| Annealing | 55 | 0:15 | |
| Extension | 72 | 2:06 | |
| Extension | 72 | 5:00 | 1 |
| Hold | 10 | — | |

A break in the ampicillin/carbenicillin cassette was introduced in error-prone PCRs resulting in two backbone fragments (i.e., pET22 (b)+ vector) and one fragment coding for a truncated or full enzyme. Mutations within the full-length fragment were introduced by varying the concentration of MnCl₂ during PCR amplification. The insert PCR conditions were as follows: Standard Taq Buffer 1×, 200 mM dNTPs each, 0.5 μM of primer NSS005, 0.5 μM of primer HR1-V2, and 0.08 U/μL of Taq polymerase, and 200-400 μM MnCl₂. Upon completion of PCRs, the remaining template was digested with DpnI (1 μL). Thermocycler Conditions for Insert PCR are outlined in Table 3. The backbone PCR conditions were as follows: Phusion GC Buffer 1×, 5% DMSO, 200 mM dNTPs each, 0.5 μM of forward primer, 0.5 μM reverse primer, and 0.02 U/μL of Phusion polymerase. Backbone fragment 1 (BB1) uses primers Amp_int_forward and NSS007. Backbone fragment 2 (BB2) uses primers HF1-V2 and Amp_int_reverse. Primer sequences for NSS005, NSS007, HR1-V2, HF1-V2, Amp_int_forward, and Amp_int_reverse are disclosed in Table 2. Thermocycler conditions for BB1/BB2 are outlined in Table 4. Upon completion of PCRs, the remaining template was digested with DpnI (1 μL). Gel purification was performed using gel electrophoresis (1% agarose gel containing a SYBR Gold nucleic acid gel stain) and DNA was visualized on a blue transilluminator. DNA was isolated using a Zymoclean DNA gel recovery kit. The purified PCR product was then assembled using the Gibson assembly protocol known in the art (Nature Methods 2009, 6, 343-345).

TABLE 2
Primers Used for PCR Metagenesis and Amplification
Primers used for PCR Mutagenesis and Amplification

| | SEQ ID | |
|---|---|---|
| Primer | NO: | Sequence (5′ to 3′) |
| NSS005 | 11 | AACTTTAAGAAGGAGATATA |
| | | CATATGACAATTAAAGAAAT |
| | | GCCTCAGCCA |
| NSS007 | 12 | TGGCTGAGGCATTTCTTTAA |
| | | TTGTCATATGTATATCTCCTT |
| | | CTTAAAGTT |
| HR1-V2 | 13 | CTTTTTTAGCAGACTGTTCA |
| | | GTGCTAGGTGAAGGAATACC |
| HF1-V2 | 14 | GGTATTCCTTCACCTAGCAC |
| | | TGAACAGTCTGCTAAAAAAG |
| Amp_int_forward | 15 | GCTAACCGCTTTTTTGCACA |
| | | ACATG |
| Amp_int_reverse | 16 | TTGTGCAAAAAAGCGGTTAG |
| | | CTCC |

TABLE 3
Thermocycler Conditions for Insert PCR
Thermocycler Conditions for Insert PCR

| Step | Temperature (ºC) | Time (min:sec) | Number of Cycles |
|---|---|---|---|
| Initial | 98 | 5:00 | 1 |
| Denaturation | | | |
| Denaturation | 98 | 00:30 | 30 |
| Annealing | 64 | 00:30 | |
| Extension | 72 | 1:30 | |
| Extension | 72 | 5:00 | 1 |
| Hold | 10 | — | |

TABLE 4
Thermocycler Conditions for BB1/BB2
Thermocycler Conditions for BB1/BB2

| Step | Temperature (ºC) | Time (min:sec) | Number of Cycles |
|---|---|---|---|
| Initial | 98 | 00:30 | 1 |
| Denaturation | | | |
| Denaturation | 98 | 00:10 | 5 |
| Annealing | 98 to 64 at 1 ºC/sec | 00:15 | |
| Extension | 72 | 2:06 | |
| Denaturation | 98 | 0:10 | 25 |
| Annealing | 64 | 0:15 | |
| Extension | 72 | 2:06 | |
| Extension | 72 | 5:00 | 1 |
| Hold | 10 | — | |

### Recombination Via Staggered Extension Process

Staggered extension process (StEP) PCR was performed using plasmids of the variants to be recombined in equimolar fashion and using this mixture as the template DNA. The StEP PCR was then conducted by varying the annealing temperature and by using a shorter extension time following a standard protocol (Nature Biotechnology 1998, 16, 258-261 and Nature Protocols 2006, 1, 1865-1871) as understood in the art. The PCR conditions were as follows: Standard Taq Buffer 1×, 200 mM dNTPs each, 0.5 μM of primer NSS005, 0.5 μM of primer HR1-V2, and 0.08 U/μL of Taq polymerase. The thermocycler conditions for recombination for the StEP insert are disclosed in Table 5. Amplification of the recombined fragment was conducted using a Phusion PCR protocol. The PCR conditions were as follows: Phusion GC Buffer 1×, 200 mM dNTPs each, 0.5 μM of primer NSS005, 0.5 μM of primer HR1-V2, and 0.02 U/μL of Phusion polymerase. Thermocycling conditions for amplification of the StEP insert are disclosed in Table 6. Upon completion of PCRs, the remaining template was digested with DpnI (1 μL). The recombined insert was purified by purified gel electrophoresis (1% agarose gel containing a SYBR Gold nucleic acid gel stain) and DNA was visualized on a blue transilluminator. The recombined insert library was purified and inserted into the pET22 (b)+ vector using a Gibson assembly (Nature Methods 2009, 6, 343-345). with BB1/BB2 fragments as understood in the art.

TABLE 5
Thermocycler Conditions for Recombination for StEP Insert
Thermocycler Conditions for Recombination of StEP Insert

| Step | Temperature (ºC) | Time (min:sec) | Number of Cycles |
|---|---|---|---|
| Initial | 95 | 5:00 | 1 |
| Denaturation | | | |
| Denaturation | 95 | 0:10 | 120 |
| Annealing | 55 | 0:05 | |
| Extension | Gradient (50 → | 0:05 | |
| | 72 °C) | | |
| Hold | 10 | — | |

TABLE 6
Thermocycler Conditions for Amplification of StEP Insert
Thermocycler Conditions for Amplification of StEP Insert

| Step | Temperature (°C) | Time (min:sec) | Number of Cycles |
|---|---|---|---|
| Initial | 98 | 5:00 | 1 |
| Denaturation | | | |
| Denaturation | 98 | 00:30 | 30 |
| Annealing | 64 | 00:30 | |
| Extension | 72 | 1:30 | |
| Extension | 72 | 5:00 | 1 |
| Hold | 10 | — | |

### Transformation and Isolation of Plasmids/Gibson Products

T7 Express Competent Escherichia coli cells were utilized for all experiments. Plasmids were mixed with competent cells on ice in PCR tubes. The mixture was kept on ice for 30 min, after which time transformation was accomplished by heat shocking the mixture in a 42° C. water bath for exactly 10 seconds. After a 5 min recovery on ice, the cells were diluted with SOC medium and plated on Luria-Bertani medium supplemented with carbenicillin (1 mg/mL) agar plates.

Plasmids were isolated from stationary-phase cultures by miniprep (Qiagen) and sequencing was performed by Laragen, Inc. (Culver City, CA) using a T7 promoter and HR1-V2 terminator primers

### Small-Scale Enzymatic Screens

For small-scale biocatalytic reactions using cell lysate, reaction vessels were either individual snap-cap 2.0 mL microtubes or 96-well plates constructed from individual 1.0 mL autosampler neckless shell vials. The 96-well plates of individual shell vials were constructed by inserting 1.0 mL autosampler neckless shell vials into a 96-well microtiter plate using a USA Scientific 1000 μL pipette tip rack as an alignment guide. The snap-cap vials were sealed by individual capping after the addition of all biocatalytic reaction components. The 96-well shell vial plates were sealed by capping with an additional 96-well microtiter plate and centrifugation (3000 g, 1 min, 25° C.).

After a single-site saturation mutagenesis (SSM), error-prone PCR (epPCR), or staggered extension process (StEP) library was generated, 84 single colonies were randomly picked and cultured in 400 mL of LB medium with 0.1 mg/mL carbenicillin (LBcarb) in a sterilized 96-well culture plate. The plate typically contained eight wells inoculated with single colonies expressing the parent enzyme, two sterile wells, and two wells inoculated with single colonies expressing Tm9D8*. The cultures were covered with a microporous film and grown at 37° C., 220 rpm, and 80% relative humidity for 12-16 h. A separate, sterilized 96-well culture plate was filled with 930 mL of Terrific Broth medium containing 0.1 mg/mL carbenicillin (TBcarb). The plate with TBcarb was inoculated with the LBcarb preculture (20 mL/well) and incubated at 37° C., 220 rpm, and 80% relative humidity for 3 h. The plate was then cooled on ice for 30 min, induced with 0.5 mM IPTG, 1 mM ALA, and X FeCl3/trace metals master mix (final concentrations), and then expressed at 22° C. and 220 rpm for 16-22 h. After expression, the cells were pelleted (4500 g, 15 min, 4° C.) via centrifugation and the supernatant was discarded. The pelleted cells were sealed with a sealing foil and stored in a −20° C. freezer for at least 16 h prior to lysis.

The 96-well plates containing frozen cell cultures were thawed for 10 min at 25° C. and the pelleted cells were then resuspended in lysis buffer (400 μL/well) containing 1 mg/mL Lysozyme, 0.07 mg/mL DNAse, and 2 mM MgCl2. The cells were then lysed at 37° C. and 160 rpm for 1 h. Cell debris was then pelleted by centrifugation (4500 g, 15 min, 4° C.). The biocatalytic reaction vessels were then charged with the NADPH cofactor regeneration system (12.5 μL/vessel) or NADPH (12.5 μL/vessel) to give final reaction concentrations of 0.5 mM NADP+, 40 mM glucose-6-phosphate, and 2 U/mL of glucose-6-phosphate dehydrogenase or 10 mM NADPH. A 280 mM substrate stock was prepared by dissolving the appropriate substrate in the specified cosolvent. The cell lysate was then added (325 μL/reaction) to the reaction vessel, followed by the substrate (12.5 μL/reaction) to give a final concentration of 10 mM substrate. The reaction vessels were sealed immediately after the addition of substrate and shaken at 800 rpm for 4 h at 25° C. After the 4 h reaction, the reaction vessels were unsealed and 5 mM 1,3,5-trimethoxybenzene or 5 mM D4 in ethyl acetate was added (400-450 μL/well) quickly. The organic and aqueous phases were mixed by using a vortex mixer (microtubes) or by pipetting the mixture up and down with a multi-channel pipette. Phase separation in microtubes was facilitated by centrifugation at 14,000 g and 4° C. for 15 min. The 96-well shell vial plates were sealed with sealing foil and centrifuged at 4000 g and 4° C. for 15 min. Aliquots of the organic phase (200 μL/reaction) were then transferred to individual 400 μL flat bottom glass inserts in screw cap 2 ml vials which were sealed with red sil septum screw cap. Analysis was performed using GC/MS with product concentrations and yield determined by response relative to the 1,3,5-trimethoxybenzene internal standard using a calibration curve.

### Determination of Hemeprotein Concentration

Hemeprotein concentration determined for purified protein and cell lysate by performing a CO-binding assay. Hemeprotein solution was added to a clear plastic flat-bottomed 96-well plates (180 μL/well) in 3-6 replicates per protein. A solution of 300 mM sodium diothionite in 1 M pH 8 potassium phosphate buffer (20 μL/well) was then added to the wells. Absorbance was then measured at 450 nm and 490 nm using a multimode microplate reader. The well plate was then placed in a CO-chamber. The atmosphere in the chamber was evacuated with a vacuum pump and the chamber was refilled to atmospheric pressure with CO. The well plate was incubated in the CO atmosphere for 30 min, then measured again using the microplate reader at 450 nm and 490 nm. Beer's law was used to determine the hemoprotein concentration of the solution using the ΔA450-490 between the CO-bound and reduced samples, the ε450-490 value of 0.091, dilution factor of 1.1, and the pathlength of 0.74 cm.

### Protein Purification Procedure

For purification, cell pellets were frozen at −20° C. for at least 24 hours. Cells were thawed and resuspended in binding buffer (20 mM Tris·HCl, 100 mM sodium chloride, 20 mM imidazole, pH 7.0, ~5 mL/g wet cells) and lysed by sonication (QSonica Q500 sonicator, 25% amplitude, 33% duty cycle, 2 minutes). The lysate was clarified by centrifugation (4500 g, 10 min) followed by filtration (0.20 μm syringe filter). The protein was purified using an purifier with an HP column, eluting with a gradient of 20-500 mM imidazole. Fractions containing the protein of interest were pooled. Fractions containing purified enzyme were pooled and concentrated with repeated centrifugation and dilution in tris buffer (0.1 M, pH 7) in an ultra-centrifugal filter (10 kDa molecular weight cutoff). The proteins were concentrated to a final concentration of 5-100 μM (as defined below). The concentrated protein was divided into aliquots (50-100 μL), flash frozen on powdered dry ice, and stored at −80° C. Protein concentration was determined by a CO-binding assay.

### Preparation Example X1: Small-Scale Enzymatic Reactions for Lineage Validation

Validation of enzymatic activity was performed as follows. E. coli transformed with pET22b(+) constructs encoding P450s were grown at 37° C. and 220 rpm in 5 mL LBcarb medium for 16-20 h. Subsequently, 0.5 mL of this preculture was used to inoculate 50 ml of TBcarb medium in a sterile 125-mL Erlenmeyer flask covered with sterilized aluminum foil. The culture was incubated at 37° C. and shaken at 220 rpm for approximately 2-4 h until the optical cell density at 600 nm (OD600) was 0.7-0.9. Then, the expression culture was cooled in an ice bath for 30 min, induced with 0.5 mM IPTG, 1 mM ALA, and 3.5 μM FeCl₃/trace metals master mix (final concentrations), and then expressed at 22° C. and 220 rpm for 16-22 h. After expression, the cultures were transferred to tared 50-mL Falcon tubes and pelleted (4500 g, 15 min, 4° C.) via centrifugation and the supernatant was discarded. The pelleted cells were stored in a −20° C. freezer for at least 16 h prior to lysis in the sealed Falcon tube.

The Falcon tubes containing frozen cell cultures were thawed for 10 min at 25° C. and the pelleted cells were then resuspended in lysis buffer (4 mL/1 g of pellet) containing 1 mg/ml Lysozyme, 0.07 mg/mL DNAse, and 2 mM MgCl2. The cells were then lysed at 37° C. and 180 rpm for 1 h. The lysed cultures were then transferred to 2.0 mL microtubes and centrifuged at 14000 g and 4° C. for 15 min. The supernatant lysates were reconstituted in 15-mL Falcon tubes. NADPH cofactor regeneration system (12.5 μL/vessel) or NADPH (12.5 μL/vessel) was then added to 2.0 mL microtubes to give final reaction concentrations of 0.5 mM NADP+, 40 mM glucose-6-phosphate, and 2 U/mL of glucose-6-phosphate dehydrogenase or 10 mM NADPH. A 280 mM substrate stock was prepared by dissolving the appropriate substrate in the specified cosolvent. The cell lysate was then added (325 μL/reaction) to the reaction vessel, followed by the substrate (12.5 μL/well) to give a final concentration of 10 mM substrate. The microtubes were sealed immediately after the addition of substrate and shaken at 800 rpm for 4 h at 25° C. After the 4 h reaction, the reaction vessels were unsealed and 5 mM 1,3,5-trimethoxybenzene in ethyl acetate was added (400-450 μL/reaction) quickly. The organic and aqueous phases were mixed by using a vortex mixer. Phase separation was facilitated by centrifugation at 14,000 g and 4° C. for 15 min. Aliquots of the organic phase (200 μL/reaction) were then transferred to individual 400 μL flat bottom glass inserts in screw cap 2 ml vials which were sealed with red sil septum screw cap. Analysis was performed using GC/MS with product concentrations and yield determined by response relative to the 1,3,5-trimethoxybenzene internal standard using a calibration curve. Superior variants were selected for subsequent mutagenesis by comparing product yields and product yields relative to estimated protein concentration. Hemeprotein concentration was determined by performing a CO-binding assay on the cell lysate.

### Preparation Example X2: Validation of Lineage for LSiIOx4 to LSiIOx5 Evolution

LBcarb cultures of unvalidated variants (80 mL/well) with improved activity which were identified from the LSiIOx4 error-prone PCR library were used to inoculate 24-well plates containing 3.75 mL of TBcarb in quadruplicate. In each plate, four wells were used as sterile controls, two wells were used as TM9D8* negative controls, and four wells were used for parent enzyme, LSILOX4. The cultures were covered with a microporous film and grown at 37° C., 205 rpm, and 80% relative humidity for 3 h. The plate was then cooled on ice for 30 min, induced with 0.5 mM IPTG, 1 mM ALA, and 3. μM FeCl₃/trace metals master mix, and then expressed at 22° C. and 205 rpm for 16-22 h. After expression, the cells were pelleted (4500 g, 15 min, 4° C.) via centrifugation and the supernatant was discarded. The pelleted cells were sealed with a sealing foil and stored in a −20° C. freezer for at least 16 h prior to lysis.

The 24-well plates containing frozen cell cultures were thawed for 10 min at 25° C. and the pelleted cells were then resuspended in lysis buffer (1.1 mL/well) containing 1 mg/ml Lysozyme, 0.07 mg/mL DNAse, and 2 mM MgCl2. The cells were then lysed at 37° C. and 135 rpm for 1 h. Cell debris was then pelleted by centrifugation (4500 g, 15 min, 4° C.). 2.0 mL microtubes were then charged with the NADPH cofactor regeneration system (25 mL/reaction) to give final reaction concentrations of 0.5 mM NADP+, 40 mM glucose-6-phosphate, and 2 U/mL of glucose-6-phosphate dehydrogenase or 10 mM NADPH. A 280 mM substrate stock was prepared by dissolving the L3 in the ethanol. The cell lysate was then added (650 mL/reaction) to the reaction vessel, followed by the substrate (25 mL/reaction) to give a final concentration of 10 mM substrate. The reaction vessels were sealed immediately after the addition of substrate and shaken at 800 rpm for 4 h at 25° C. After the 4 h reaction, the reaction vessels were unsealed and 5 mM 1,3,5-trimethoxybenzene in ethyl acetate was added (800 mL/reaction) quickly. The organic and aqueous phases were mixed by using a vortex mixer. Phase separation in microtubes was facilitated by centrifugation at 14,000 g and 4° C. for 15 min. Aliquots of the organic phase (200 mL/reaction) were then transferred to individual 400 mL flat bottom glass inserts in screw cap 2 ml vials which were sealed with red sil septum screw cap. Analysis was performed using GC/MS with product concentrations and yield determined by response relative to the 1,3,5-trimethoxybenzene internal standard using a calibration curve.

### Preparation Example X3: Small-Scale Enzymatic Reactions with Purified Protein

NADPH (12.5 μL/vessel) was added to 2.0 mL microtubes to give final reaction concentrations of 10 mM NADPH. A 320 mM substrate stock was prepared by dissolving the appropriate substrate in 200 proof ethanol. Purified protein and buffer to a total of 375 μL were added to the reaction vessel to give a final protein concentration of 5 μM, followed by the substrate (12.5 μL/well) to give a final concentration of 10 mM substrate. The microtubes were sealed immediately after the addition of substrate and shaken at 800 rpm for 4 h at 25° C. After the 4 h reaction, the reaction vessels were unsealed and 5 mM D4 in ethyl acetate was added (400 μL/reaction) quickly. The organic and aqueous phases were mixed by using a vortex mixer. Phase separation was facilitated by centrifugation at 14,000 g and 4° C. for 15 min. Aliquots of the organic phase (200 μL/reaction) were then transferred to individual 400 μL flat bottom glass inserts in screw cap 2 ml vials which were sealed with red sil septum screw cap. Analysis was performed using GC/MS with product concentrations and yield determined by response relative to the D4 internal standard using a calibration curve.

TABLE 7
Lineage reactions with hexamethyldisiloxane

| | | | | Percent of | | | Percent |
|---|---|---|---|---|---|---|---|
| | | MM^{CH2OH} | MM^{CH2OH} | total | MM^{OH} | MM^{OH} | of total |
| | | Peak | Yield | product | Peak | Yield | product |
| Variant | Example | Area | (mM) | (MM^{CH2OH}) | Area | (mM) | (MM^{OH}) |
| P450_{BM3} | Comparative | 0 | 0 | 0 | 0 | 0 | 0 |
| WT (0) | 1 | | | | | | |
| P450_{BM3} | Comparative | 0 | 0 | 0 | 0 | 0 | 0 |
| WT (0) | 2 | | | | | | |
| LSilOx1 | 1 | 5907 | 0.0200 | 25.0 | 13558 | 0.0600 | 75.0 |
| LSilOx1 | 2 | 4655 | 0.0200 | 28.6 | 10850 | 0.0500 | 71.4 |
| LSilOx2 | 3 | 14117 | 0.0500 | 41.7 | 15250 | 0.0700 | 58.3 |
| LSilOx2 | 4 | 14027 | 0.0600 | 46.2 | 13542 | 0.0700 | 53.8 |
| LSilOx3 | 5 | 27310 | 0.100 | 45.5 | 25091 | 0.120 | 54.5 |
| LSilOx3 | 6 | 27292 | 0.100 | 45.5 | 25141 | 0.120 | 54.5 |
| LSilOx4 | 7 | 30264 | 0.120 | 50.0 | 22746 | 0.120 | 50.0 |
| LSilOx4 | 8 | 27503 | 0.110 | 47.8 | 23635 | 0.120 | 52.2 |

Reaction conditions are as described in Preparation Example X3:10 mM L2, purified P450BM3 variant, 10 mM NADPH, 100 mM pH 7 Tris buffer, 3.6% EtOH, 25° C., 4 h, aerobic.

### Hexamethyldisiloxane Lineage Reaction

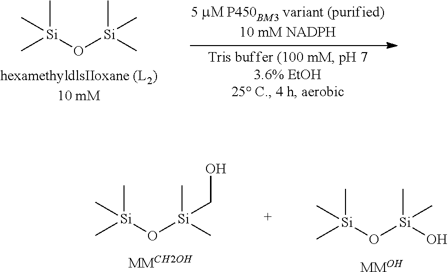

TABLE 8
Lineage validation reactions with octamethyltrisiloxane

| | | | | | Percent | | | Percent | | | |
|---|---|---|---|---|---|---|---|---|---|---|---|
| | | 1,3,5- | | | of | | | of | | | Percent |
| | | TMB | MDM^{CH2OH} | MDM^{CH2OH} | total | MDM^{OH} | MDM^{OH} | total | MM^{OH} | MM^{OH} | of total |
| | | Peak | Peak | Yield | product | Peak | Yield | product | Peak | Yield | product |
| Variant | Ex | Area | Area | (mM) | (MDM^{CH2OH}) | Area | (mM) | (MDM^{OH}) | Area | (mM) | (MM^{OH}) |
| LSilOx4 | 9 | 1100094 | 2046 | 0.00563 | 24.0 | 12308 | 0.0126 | 53.8 | 1086 | 0.00522 | 22.2 |
| LSilOx4 | 10 | 1206964 | 2221 | 0.00557 | 20.4 | 16653 | 0.0156 | 57.1 | 1402 | 0.00614 | 22.5 |
| LSilOx4 | 11 | 1191801 | 2705 | 0.00687 | 23.6 | 15773 | 0.0149 | 51.4 | 1633 | 0.00724 | 24.9 |
| LSilOx4 | 12 | 1168236 | 2335 | 0.00605 | 21.1 | 16363 | 0.0158 | 55.2 | 1494 | 0.00676 | 23.6 |
| LSilOx4 | 13 | 1103289 | 2221 | 0.00609 | 21.0 | 16083 | 0.0165 | 56.6 | 1363 | 0.00653 | 22.5 |
| LSilOx4 | 14 | 1190968 | 2495 | 0.00634 | 20.6 | 18318 | 0.0174 | 56.5 | 1582 | 0.00702 | 22.8 |
| LSilOx4 | 15 | 1242988 | 3275 | 0.00797 | 23.1 | 20568 | 0.0187 | 54.1 | 1856 | 0.00789 | 22.8 |
| LSilOx4 | 16 | 1294910 | 3303 | 0.00772 | 21.9 | 23054 | 0.0201 | 57.1 | 1808 | 0.00738 | 21.0 |
| LSilOx4 | 17 | 1269725 | 3678 | 0.00877 | 24.1 | 22125 | 0.0197 | 54.2 | 1895 | 0.00789 | 21.7 |
| LSilOx4 | 18 | 1284069 | 3379 | 0.00796 | 21.8 | 23202 | 0.0204 | 55.7 | 2004 | 0.00825 | 22.5 |
| LSilOx4 | 19 | 1206905 | 3137 | 0.00787 | 20.8 | 23866 | 0.0223 | 59.04 | 1741 | 0.00762 | 20.2 |
| LSilOx5 | 20 | 1416425 | 2361 | 0.00505 | 6.48 | 86917 | 0.0693 | 88.9 | 4493 | 0.00358 | 4.60 |
| LSilOx5 | 21 | 1204577 | 2158 | 0.00542 | 5.50 | 94274 | 0.0884 | 89.6 | 5200 | 0.00487 | 4.94 |
| LSilOx5 | 22 | 1093112 | 1848 | 0.00512 | 4.81 | 92927 | 0.0960 | 90.1 | 5230 | 0.00540 | 5.07 |

Reaction conditions are as described in Preparation Example X2:10 mM L3, P450BM3 variant in lysate, 40 mM glucose-6-phosphate, 2 U/mL glucose-6-phosphate-dehydrogenase, 0.5 mM NADP+, 100 mM pH 7 Tris buffer, 3.6% EtOH, 25° C., 4 h, aerobic.

### Octamethyltrisiloxane Lineage Reaction

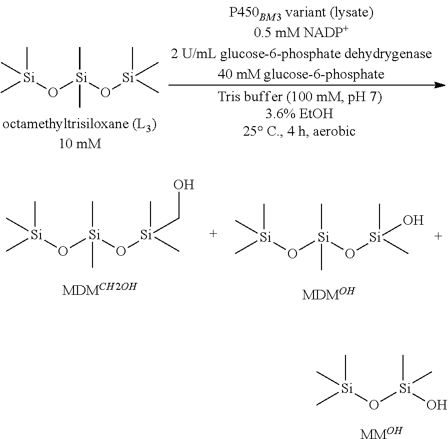

TABLE 9
Lineage validation reactions with octamethylcylcotetrasiloxane

| | | | D₃D^{OH} Peak | D₃D^{OH} Yield |
|---|---|---|---|---|
| Variant | Example | 1,3,5-TMB Peak Area | Area | (mM) |
| LSilOx4 | 23 | 935820 | 8455 | 0.00835 |
| LSilOx4 | 24 | 929143 | 10673 | 0.0106 |
| LSilOx4 | 25 | 919988 | 8007 | 0.00804 |
| LSilOx5 | 26 | 949383 | 18528 | 0.0180 |
| LSilOx5 | 27 | 874224 | 18675 | 0.0197 |

Reaction conditions are as described in Preparation Example X1:10 mM D4, P450BM3 variant in lysate, 40 mM glucose-6-phosphate, 2 U/mL glucose-6-phosphate-dehydrogenase, 0.5 mM NADP+, 100 mM pH 7 Tris buffer, 3.6% EtOH, 25° C., 4 h, aerobic.

### Octamethylcyclotetrasiloxane Lineage Validation Reaction

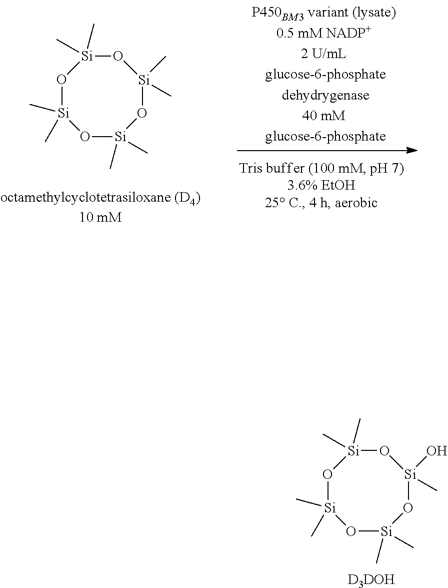

TABLE 10
Brook rearrangement experiments using hexamethyldisiloxane

| | | | | Percent of | | | Percent |
|---|---|---|---|---|---|---|---|
| | | MM^{CH2OH} | MM^{CH2OH} | total | MM^{OH} | MM^{OH} | of total |
| | | Peak | Yield | product | Peak | Yield | product |
| Variant | Example | Area | (mM) | (MM^{CH2OH}) | Area | (mM) | (MM^{OH}) |
| P450_{BM3} | Comparative 3 | 0 | 0 | 0 | 0 | 0 | 0 |
| WT (0) | | | | | | | |
| P450_{BM3} | Comparative 4 | 0 | 0 | 0 | 0 | 0 | 0 |
| WT (0) | | | | | | | |
| Hemin | 28 | 0 | 0 | 0 | 0 | 0 | 0 |
| Chloride | | | | | | | |
| Hemin | 29 | 0 | 0 | 0 | 0 | 0 | 0 |
| Chloride | | | | | | | |
| Hemin | 30 | 0 | 0 | 0 | 0 | 0 | 0 |
| Chloride + | | | | | | | |
| BSA | | | | | | | |
| Hemin | 31 | 0 | 0 | 0 | 0 | 0 | 0 |
| Chloride + | | | | | | | |
| BSA | | | | | | | |
| BSA | 32 | 0 | 0 | 0 | 0 | 0 | 0 |
| BSA | 33 | 0 | 0 | 0 | 0 | 0 | 0 |
| LSilOx2 | 34 | 2596 | 0.0229 | 25.0 | 6353 | 0.0686 | 75.0 |
| LSilOx2 | 35 | 1097 | 0.0114 | 14.3 | 6250 | 0.0686 | 85.7 |
| LSilOx4 | 36 | 1590 | 0.0114 | 5.26 | 21566 | 0.206 | 94.7 |
| LSilOx4 | 37 | 1343 | 0.0114 | 5.56 | 20489 | 0.194 | 94.4 |

Reaction conditions are as described in Preparation Example X3:10 mM L2, 5 μM purified P450BM3 variant or control, 10 mM NADPH, 100 mM pH 7 Tris buffer, 3.6% EtOH, 25° C., 4 h, aerobic.

### Hexamethyldisiloxane Brook Rearrangement Reaction

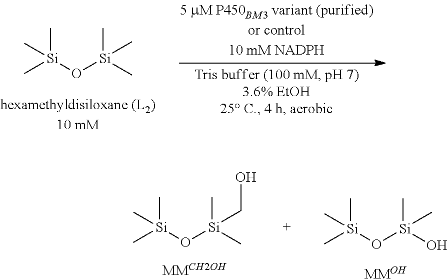

TABLE 11
Brook rearrangement experiments using MM^{CH2OH}

| Control or Protein Variant | | MM^{OH} | |
|---|---|---|---|
| (generation number) | Example | Peak Area | MM^{OH} Yield (mM) |
| P450_{BM3} WT (0) | Comparative | 2117 | 0.0229 |
| | 5 | | |
| P450_{BM3} WT (0) | Comparative | 2174 | 0.0229 |
| | 6 | | |
| Hemin Chloride | 38 | 0 | 0 |
| Hemin Chloride | 39 | 0 | 0 |
| Hemin Chloride + BSA | 40 | 0 | 0 |
| Hemin Chloride + BSA | 41 | 0 | 0 |
| BSA | 42 | 0 | 0 |
| BSA | 43 | 0 | 0 |
| LSilOx2 | 44 | 16044 | 0.171 |
| LSilOx2 | 45 | 12287 | 0.137 |
| LSilOx4 | 46 | 33833 | 0.343 |
| LSilOx4 | 47 | 31913 | 0.331 |

Reaction conditions are as described in Preparation Example X3:10 mM L2, 5 μM purified P450BM3 variant or control, 10 mM NADPH, 100 mM pH 7 Tris buffer, 3.6% EtOH, 25° C., 4 h, aerobic.

Brook Rearrangement Reaction using MM^{CH2OH}

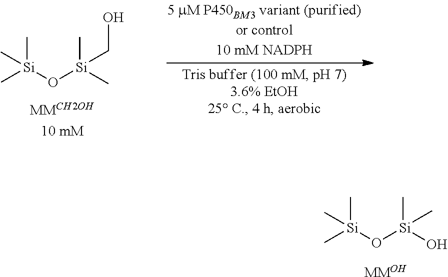

TABLE 12
Cosolvent facilitates enzymatic reaction.

| | | | | | Percent | | | Percent | | | Percent |
|---|---|---|---|---|---|---|---|---|---|---|---|
| | | 1,3,5- | | | of | | | of | | | of |
| | | TMB | MDM^{CH2OH} | MDM^{CH2OH} | total | MDM^{OH} | MDM^{OH} | total | MM^{OH} | MM^{OH} | total |
| Co- | Ex- | Peak | Peak | Yield | product | Peak | Yield | product | Peak | Yield | product |
| solvent | ample | Area | Area | (mM) | (MDM^{CH2OH}) | Area | (mM) | (MDM^{OH}) | Area | (mM) | (MM^{OH}) |
| THF | 48 | 919373 | 6957 | 0.0229 | 8.98 | 128694 | 0.158 | 61.9 | 12905 | 0.0742 | 29.1 |
| THF | 49 | 891299 | 4671 | 0.0159 | 6.31 | 128346 | 0.163 | 64.7 | 12303 | 0.0729 | 29.0 |
| THF | 50 | 969020 | 5408 | 0.0169 | 7.16 | 130042 | 0.151 | 64.2 | 12382 | 0.0675 | 28.6 |
| MeCN | 51 | 953495 | 4270 | 0.0136 | 5.26 | 142514 | 0.169 | 65.5 | 13603 | 0.0754 | 29.3 |
| MeCN | 52 | 930450 | 3620 | 0.0118 | 4.73 | 125424 | 0.152 | 61.1 | 14953 | 0.0849 | 34.1 |
| MeCN | 53 | 960368 | 2447 | 0.00771 | 3.45 | 118004 | 0.139 | 62.0 | 14035 | 0.0772 | 34.5 |
| EtOH | 54 | 983704 | 5401 | 0.0166 | 7.17 | 109348 | 0.125 | 54.2 | 16665 | 0.0895 | 38.6 |
| EtOH | 55 | 969440 | 3395 | 0.0106 | 5.33 | 94984 | 0.111 | 55.6 | 14231 | 0.0776 | 39.0 |
| EtOH | 56 | 959671 | 2696 | 0.00850 | 5.04 | 86176 | 0.101 | 60.1 | 10666 | 0.0587 | 34.8 |
| No | 57 | 923032 | 1841 | 0.00604 | 7.36 | 36293 | 0.0444 | 54.2 | 5510 | 0.0315 | 38.5 |
| Cosolvent | | | | | | | | | | | |
| No | 58 | 902906 | 950 | 0.00318 | 2.50 | 56474 | 0.0706 | 55.4 | 9156 | 0.0536 | 42.1 |
| Cosolvent | | | | | | | | | | | |
| No | 59 | 910892 | 801 | 0.00266 | 3.14 | 36625 | 0.0454 | 53.5 | 6337 | 0.0368 | 43.3 |
| Cosolvent | | | | | | | | | | | |

Reaction conditions are as described in Preparation Example X1:10 mM L3, P450BM3 variant in lysate, 40 mM glucose-6-phosphate, 2 U/mL glucose-6-phosphate-dehydrogenase, 0.5 mM NADP+, 100 mM pH 7 Tris buffer, 3.6% cosolvent or equivalent volume of buffer without cosolvent, 25° C., 4 h, aerobic.

### Cosolvent Facilitated Enzymatic Reaction

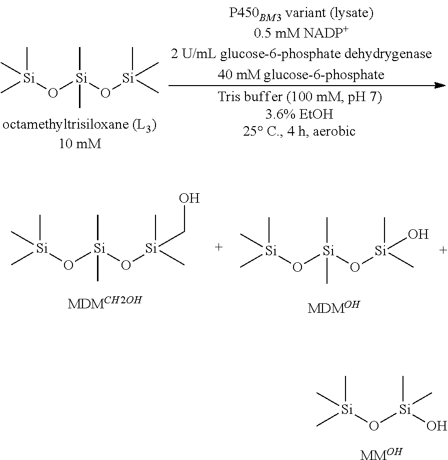

TABLE 13
Mutation Summary and Sequence of Enzyme Variants
Summary of Mutations

| | | | | Silent |
|---|---|---|---|---|
| | | Mutations | Silent | Mutations |
| | | From | Mutations | From |
| Variant | Mutations From P450_{BM3}-WT | Parent | From WT | Parent |
| P450_{BM3}-WT | — | | — | |
| LSilOx1 | V78A, A82V, F87G, P142S, | | F107F, | |
| | T1751, A184V, S226R, H236Q, | | E372E | |
| | E252G, A290V, L353V, 1366V, | | | |
| | E442K | | | |
| LSilOx2 | V78A, A82V, F87G, P142S, | T327M, | F107F, | 14531 |
| | T1751, A184V, S226R, H236Q, | A328F | E372E, | |
| | E252G, A290V, T327M, A328F, | | 14531 | |
| | L353V, 1366V, E442K | | | |
| LSilOx3 | D34G, V78A, A82V, F87G, | D34G, | F107F, | L249L, |
| | 1122T, P142S, T1751, A184V, | 1122T | L249L, | E337E, |
| | S226R, H236Q, E252G, A290V, | | E337E, | L341L, |
| | T327M, A328F, L353V, 1366V, | | L341L, | E380E, |
| | E442K | | E372E, | P386P |
| | | | E380E, | |
| | | | P386P, | |
| | | | 14531 | |
| LSilOx4 | D34G, V78A, A82V, F87G, | G252E | F107F, | L249L, |
| | 1122T, P142S, T1751, A184V, | (reversion | E372E, | E337E, |
| | S226R, H236Q, A290V, T327M, | to WT) | 14531 | L341L, |
| | A328F, L353V, 1366V, E442K | | | E380E, |
| | | | | P386P (all |
| | | | | reversions |
| | | | | to WT) |
| LSilOx5 | D34G, V78A, A82V, F87G, | F328S | F107F, | D422D |
| | 1122T, P142S, T1751, A184V, | | E372E, | |
| | S226R, H236Q, A290V, T327M, | | D422D, | |
| | A328S, L353V, 1366V, E442K | | 14531 | |
| LSilOx6 | D34G, V78A, A82V, F87G, | F165L | F107F, | |
| | 1122T, P142S, T1751, F165L, | | E372E, | |
| | A184V, S226R, H236Q, A290V, | | D422D, | |
| | T327M, A328S, L353V, 1366V, | | 1453\| | |
| | E442K | | | |
| LSilOx7 | D34G, Y51V, V78A, A82V, F87G, | Y51V, | F107F, | |
| | 1122T, P142S, T1751, F165L, | V184M | E372E, | |
| | A184M, S226R, H236Q, A290V, | | D422D, | |
| | T327M, A328S, L353V, 1366V, | | 14531 | |
| | E442K | | | |
| CSilOx1 | D34G, V78A, A82V, F87G, N95S, | N95S, | F107F, | H285H |
| | 1122T, P142S, T1751, A184V, | D214G, | H285H, | |
| | D214G, S226R, H236Q, A290V, | T438S | E372E, | |
| | T327M, A328S, L353V, 1366V, | | D422D, | |
| | T438S, E442K | | 14531 | |
| CSilOx2 | D34G, S72G, V78A, A82V, | S72G, | F107F, | |
| | G85A, F87G, N95S, 1122T, | G85A | H285H, | |
| | P142S, T1751, A184V, D214G, | | E372E, | |
| | S226R, H236Q, A290V, T327M, | | D422D, | |
| | A328S, L353V, 1366V, T438S, | | 14531 | |
| | E442K | | | |
| CSilOx3 | D34G, R47G, S72G, V78A, | R47G | F107F, | |
| | A82V, G85A, F87G, N95S, | | H285H, | |
| | 1122T, P142S, T1751, A184V, | | E372E, | |
| | D214G, S226R, H236Q, A290V, | | D422D, | |
| | T327M, A328S, L353V, 1366V, | | 14531 | |
| | T438S, E442K | | | |

1. A method of preparing an organosilicon compound having at least one silicon-bonded carbinol group and/or at least one silanol group, said method comprising:
preparing a reaction mixture by combining a cytochrome P450 variant that facilitates the oxidation of a silicon-bonded hydrocarbyl group to a silicon-bonded carbinol group or a silanol group in the presence of an oxidant, an initial organosilicon compound having at least one silicon-bonded hydrocarbyl group, and a cofactor; and
exposing the reaction mixture to an oxidant to oxidize and convert the silicon-bonded hydrocarbyl group to a silicon-bonded carbinol group or a silicon-bonded silanol group, thereby preparing the organosilicon compound having at least one silicon-bonded carbinol group and/or at least one silanol group.

2. The method of claim 1, wherein the cytochrome P450 variant comprises the nucleic acid sequence of SEQ ID NO:1 or a conservatively modified variant thereof.

3. The method of claim 1, wherein the cytochrome P450 variant comprises the nucleic acid sequence of SEQ ID NO:1 or a conservatively modified variant thereof with a mutation of at least one of T328, A329, D35, I123, G253, F329, F166, Y52, V185, N96, D215, T439, S73, G86, or R48 relative to the nucleic acid sequence of SEQ ID NO:1.

4. The method of claim 3, wherein the cytochrome P450 variant comprises: (i) a T328M mutation; (ii) an A329F mutation; (iii) a D35G mutation; (iv) an I123T mutation; (v) a G253E mutation; (vi) an F329S mutation; (v) a F166L mutation; (vi) a Y52V mutation; (vii) a V185M mutation; (viii) an N96S mutation, (ix) a D215G mutation; (x) a T439S mutation; (xi) an S73G mutation; (xii) a G86A mutation; (xiii) an R48G mutation; or (xiv) any combination of (i)-(xiii), relative to the nucleic acid sequence of SEQ ID NO:1.

5. The method of claim 1, wherein: (i) the cytochrome P450 variant comprises the nucleic acid sequence of SEQ ID NO:2 or a conservatively modified variant thereof; or (ii) the cytochrome P450 variant comprises the nucleic acid sequence of SEQ ID NO:3 or a conservatively modified variant thereof; or (iii) the cytochrome P450 variant comprises the nucleic acid sequence of SEQ ID NO:4 or a conservatively modified variant thereof; or (iv) the cytochrome P450 variant comprises the nucleic acid sequence of SEQ ID NO:5 or a conservatively modified variant thereof; or (v) the cytochrome P450 variant comprises the nucleic acid sequence of SEQ ID NO:6 or a conservatively modified variant thereof; or (vi) the cytochrome P450 variant comprises the nucleic acid sequence of SEQ ID NO:7 or a conservatively modified variant thereof; or (vii) the cytochrome P450 variant comprises the nucleic acid sequence of SEQ ID NO:8 or a conservatively modified variant thereof; or (viii) the cytochrome P450 variant comprises the nucleic acid sequence of SEQ ID NO:9 or a conservatively modified variant thereof; or (ix) the cytochrome P450 variant comprises the nucleic acid sequence of SEQ ID NO:10 or a conservatively modified variant thereof.

6. The method of claim 1, wherein the cytochrome P450 variant comprises a non-native heme cofactor.

7. The method of claim 1, wherein preparing the reaction mixture comprises combining the initial organosilicon compound with a host cell or non-human organism that expresses the cytochrome P450 variant, a lysate thereof, a purified protein thereof, or a lyophilized form thereof.

8. The method of claim 7, wherein the host cell or non-human organism is further defined as an E. coli cell.

9. The method of claim 1, wherein the initial organosilicon compound is free from silicon-bonded carbinol groups.

10. The method of claim 1, wherein the initial organosilicon compound has the general formula (I) or (II) or (III): 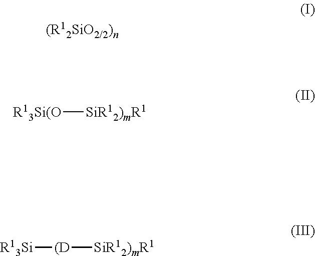
where each R¹ is an independently selected unsubstituted hydrocarbyl group or H, with the proviso that at least one of R¹ is a hydrocarbyl group, subscript n is from 3 to 8, subscript m is from 0 to 15, and each D is an independently selected divalent linking group.

11. The method of claim 10, wherein each R¹ is independently selected from alkyl groups, alkenyl groups, aryl groups, and hydrogen, with the proviso that at least one R¹ is an alkyl group, an alkenyl group, or an aryl group.

12. The method of claim 1, wherein the reaction mixture further comprises: (i) a carrier vehicle; or (ii) a buffer; or (iii) both a carrier vehicle and a buffer.

13. A method of preparing a reaction product, said method comprising:
preparing a reaction mixture by combining a cytochrome P450 variant that facilitates the oxidation of a silicon-bonded hydrocarbyl group to a functional group selected from a silicon-bonded carbinol group or a silanol group in the presence of an oxidant and an initial organosilicon compound having at least one silicon-bonded hydrocarbyl group;
exposing the reaction mixture to an oxidant to oxidize and convert the silicon-bonded hydrocarbyl group to the functional group and give an organosilicon compound having the functional group; and
reacting the functional group of the organosilicon compound with a group reactive with the functional group of the organosilicon compound, thereby preparing the reaction product.
